(19) United States
(12) Patent Application Publication
(10) Pub. No.: US 2026/0306286 A1
(43) Pub. Date: Oct. 8, 2026

(54) METHOD FOR INHIBITING DAMAGE OF LEAFLETS WHEN REDUCING A PROFILE OF A VASCULAR PROSTHESIS

(71) Applicant: MiRus LLC, Marietta, GA (US)

(72) Inventors: Ken Holloway, Marietta, GA (US); Alex Panter, Marietta, GA (US)

(21) Appl. No.: 19/571,591

(22) Filed: Mar. 19, 2026

Related U.S. Application Data
(60) Provisional application No. 63/782,176, filed on Apr. 2, 2025.
(60) Provisional application No. 63/915,192, filed on Nov. 11, 2025.

(51) Int. Cl. A61F 2/95; A61F 2/24

(52) U.S. Cl. CPC A61F 2/9522; A61F 2/2418; A61F 2/2433

(57) ABSTRACT

Crimper systems, devices, and methods for reducing the crimped profile of a medical such as, but not limited to, a prosthetic valve, and facilitate in the desired crimping of the leaflets of a prosthetic valve during the crimping of the frame of the prosthetic valve, while reducing the risk of damage to the leaflets and other components of the prosthetic valve during the crimping process.

### REFERENCED APPLICATIONS

The present application claims priority on U.S. Provisional Application Ser. Nos. 63/782,176 filed Apr. 2, 2025 and 63/915,192 filed Nov. 11, 2025, which are both fully incorporated herein by reference.

### FIELD OF DISCLOSURE

The present disclosure relates generally to methods for reducing the crimped profile of a medical device, particularly to methods for reducing the crimped profile of a prosthetic valve, and even more particularly to devices and methods for reducing the crimped profile of a prosthetic valve that inhibit or prevent damage to the leaflets of a prosthetic valve during the crimping of the frame of the prosthetic valve.

### BACKGROUND OF DISCLOSURE

Many cardiovascular devices such as expandable heart valves and the like are inserted into a patient via the vascular system of a patient and then expanded at the treatment site. These devices are typically crimped onto catheter prior to insertion into a patient. The minimum diameter to which the medical device can be crimped onto the catheter will set a limit to the size of the vascular passageway (e.g., blood vessel, etc.) to which the medical device can be inserted. Smaller crimp diameters can result in reduced damage to a vascular passageway (e.g., blood vessel, etc.) and/or organ (e.g., heart, etc.) when inserting into or to and/or placing the medical device at the treatment site. Smaller crimp diameters can also allow the medical device to be placed in smaller vascular passageways (e.g., blood vessels located in the brain, etc.).

Medical devices such as Transcatheter aortic valves (TAVs) represent a significant advancement in prosthetic valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; 12,383,399 and US Pub. No. 2020/0405482, all of which are incorporated fully herein by reference.

A TAV is designed to be compressed into a small diameter catheter, remotely placed within a patient's diseased aortic valve to take over the function of the native valve. Some TAVs are balloon-expandable, while others are self-expandable. In both cases, the TAVs are deployed within a calcified native valve and then forced permanently open and becomes the surface against which the frame is held in place by friction. TAVs can also be used to replace failing bioprosthetic or transcatheter valves, commonly known as a valve in valve procedure. Major TAVR advantages to the traditional surgical approaches include refraining from cardiopulmonary bypass, aortic cross-clamping and sternotomy which significantly reduces patients'morbidity.

TAVR involves delivery, deployment, and implantation of a crimped, framed valve within a diseased aortic valve or degenerated bioprosthesis. During the crimping of the TAV, the portion of one or more leaflets may move into the spaces in the frame and become damaged as the frame is crimped. Such damage can include a torn leaflet, the disfiguring of the shape of the leaflet, the partial or full detachment of the leaflet from the frame, etc., which damage can adversely affect the proper operation of the implanted TAV and/or reduce the life of the TAV.

Prior art arrangements used to minimize portions of the leaflets from protruding through the frame openings during the frame crimping process include the use of a compressible sleeve (e.g., compressible foam sleeve, etc.) that is positioned about the frame during the crimping of the frame. The compressible sleeve is generally placed around the outer surface of the frame, and the compressible material (e.g., foam, etc.) on the compressible sleeve is forced into one or more of the frame openings during the crimping process to inhibit or prevent one or more portions of one or more leaflets from moving into one or more openings of the frame during the crimping process. However, one limitation of using the compressible sleeve is that as the foam is compressed into the frame openings, the foam positioned in the frame openings interferes or prevents the frame from being fully crimped during the crimping process due to the foam interfering and preventing compression of the frame during the crimping process. As a result, the crimping process requires multiple crimping steps when a compressible sleeve is used, wherein the first crimping step is used to partially crimp the frame using the compressible sleeve, the second step involves removing the partially crimped prosthetic valve from the crimper, the third step involves removing the compressible sleeve from the prosthetic valve, the fourth step involves reinserting the partially crimped prosthetic valve back into the crimper, and the fifth step involves further crimping of the prosthetic valve in the crimper to obtain the fully crimped profile (smallest frame diameter) of the prosthetic valve. When the compressible sleeve is removed from the frame after the prosthetic valve has been partially crimped, the frame openings are typically small due to the frame being partially crimped, therefore large protrusions of one or more portions of the one or more leaflets into and/or through the small frame openings during second and subsequent crimping steps of the prosthetic valve are uncommon. However, during these second and subsequent crimping steps of the prosthetic valve, small portions of one or more leaflets can still slip between the reduced sized frame openings and during the further crimping of the prosthetic valve and thereby damage such portions of the leaflets. Such damage typically occurs at the edges of the frame and the outer edges of one or more of the leaflets. If one or more the leaflets are damaged during the these subsequent crimping processes of the frame of the prosthetic valve, such damage can lead to acute (tears in the leaflet leading to regurgitation) or long-term issues (Hypoattenuating Leaflet Thickening [HALT]) during the operation of the prosthetic valve at the treatment site, both of which can adversely affect the proper operation of the prosthetic valve and/or life of the prosthetic valve after implantation in a treatment site of a patient.

As such, there has been an ongoing need for an improved prosthetic valve that can a) form small crimping diameters, b) obtain crimped diameter reductions while minimizing the damage to the leaflets during the crimping process, and/or c) addresses some of the deficiencies of prior art prosthetic valves.

### SUMMARY OF THE DISCLOSURE

The present disclosure relates to crimper systems, devices, and methods that facilitate in the desired crimping of the leaflets of a prosthetic valve during the crimping of the frame of the prosthetic valve, while reducing the risk of damage to the leaflets and other components of the prosthetic valve during the crimping process. In one non-limiting embodiment, a temporary and releasable/removable barrier material (e.g., releasable/removeable tape, releasable/removable PTFE tape, etc.) is used to facilitate in the reducing or preventing of damage to one or more leaflets during the crimping of the frame of a prosthetic valve (e.g., TAV valve, aortic, mitral valve replacement, tricuspid valve replacement, pulmonary valve replacement, etc.). It has been found that the use of a temporary/removeable barrier material on a prosthetic valve is effective to inhibit or prevent damage to one or more leaflets during the crimping of the prosthetic valve; however, the proper insertion of the temporary/removeable barrier material on the prosthetic valve can be challenging due to the temporary/removeable barrier material being a challenge to manipulate about portions of the prosthetic valve, especially when the user is wearing gloves. When using the temporary/removeable barrier material releasable/removeable tape, releasable/removable PTFE tape, etc.), the temporary/removeable barrier material needs to be applied over a certain portion of the prosthetic valve, and not on other portions of the prosthetic valve since so as to prevent the temporary/removeable barrier material from interfering with the proper crimping of the prosthetic valve. Furthermore, the temporary/removeable barrier material needs to remain in place during handling of the prosthetic valve prior to being crimped and during the crimping process. Also, the temporary/removeable barrier material should be easily removable (e.g., fully removable, etc.) from the prosthetic valve after the crimping of the prosthetic valve, and the removal of the temporary/removeable barrier material should be such that the components of the prosthetic valve are not damaged during the removal of the temporary/removeable barrier material from the prosthetic valve.

In one non-limiting aspect of the present disclosure, there is provided a crimper device that is configured to reduce the outer diameter of the frame of the prosthetic valve along a longitudinal length of the prosthetic valve, wherein the crimper device reduces the frame a) uniformly along the full length of the frame, b) gradually (in a continuous manner) from i) the inflow side to the outflow side, or ii) from the outflow side to the inflow side, or c) stepwise (in a discreet manner) from i) the inflow side to the outflow side, or ii) from the outflow side to the inflow side. In either the continuous or stepwise crimping method, the inflow side portion or the outflow side portion can be partially or fully crimped prior to crimping the other portions of the frame of the prosthetic valve. In one non-limiting embodiment, the crimper device is used to reduce the outer diameter of the frame of the prosthetic valve along a longitudinal length of the prosthetic valve in a gradual or continuous manner from the inflow side to the outflow side of the prosthetic valve. In another non-limiting embodiment, the crimper device is used to reduce the outer diameter of the frame of the prosthetic valve along a longitudinal length of the prosthetic valve in stepwise manner from the inflow side to the outflow side of the prosthetic valve.

In another one non-limiting aspect of the present disclosure, there is provided a crimper device that is configured to a) uniformly crimp the frame of the prosthetic valve along the full length of the frame, b) progressively crimp a frame of the prosthetic valve from one end to the other end of the frame, c) progressively crimp a frame of the prosthetic valve from one portion to another portion of the frame, or d) batch crimping of different portions of the frame at different times and/or locations during the crimping process (e.g., crimping a front portion of the frame prior to crimping the rear portion of the frame, etc.).

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve. The one or more layers of a temporary/removeable barrier material are used to inhibit or prevent damage to one or more leaflets during the crimping of the frame. In one non-limiting embodiment, the prosthetic valve includes three leaflets that are partially or fully connected to the frame of the prosthetic valve; however, other types of prosthetic valve can include other numbers of leaflets. The leaflets can be formed of polymers, bovine pericardial tissue, bovine tissue, porcine tissue, biocompatible synthetic materials, or various other suitable natural or synthetic materials; however, other materials can be used. These leaflets are commonly a) sewn or stitched together and/or b) stitched and/or otherwise connected to the frame and/or one or more other components of the prosthetic valve (e.g., inner skirt, etc.). Prior to delivering the prosthetic valve to a treatment site, the frame of the prosthetic valve is commonly crimped from its expanded orientation to a crimped orientation onto a delivery system that can optionally include an inflatable balloon. The frame of the prosthetic valve is crimped to reduce its overall profile (reduce outer diameter) to enable passage through the patient's vasculature to the treatment site (e.g., aortic valve, mitral valve, tricuspid valve, pulmonary valve, valve in left atrial appendage, etc.). During the crimping of the frame, the leaflets are compressed, with the degree of compression that is generally inversely correlated to the degree of reduction in diameter of the frame (i.e., as the frame diameter is reduced, the degree of compression of the leaflets increases). As this compression on the leaflets occurs during the crimping process, one or more portions of one or more leaflets can be forced from the inside of the frame through one or more of the frame openings. When one or more portions of one or more leaflets protrude through one or more the frame openings, and the frame diameter is further reduced during the continued crimping process, these one or more portions of one or more leaflets that are located in and/or partially extending through one or more of the frame openings (e.g., openings formed between the struts, posts, etc. of the frame) can be damaged by the pressing, pinching, shearing, and/or tearing action of the frame on the one or more portions of one or more leaflets that are positioned in or protruding through one or more of the frame openings. The use of the temporary/removeable barrier material has been found to inhibit or prevent damage to the one or more leaflets by inhibiting or prevent the protrusion of portions of the one or more leaflets in/through the one or more frame openings during the crimping of the prosthetic valve.

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve to inhibit or prevent damage to the one or more leaflets during the crimping of the prosthetic valve, and wherein the material selected for the one or more layers of a temporary/removeable barrier material is a) flexible so that the material can be partially or forced into the frame openings to inhibit or prevent one or more portions of the one or more leaflets from moving into and/or through one or more openings in the frame during the crimping of the prosthetic valve, b) thin enough so that the one or more layers of material can be partially or fully forced into the frame openings in the frame during the crimping of the prosthetic valve, c) robust enough or strong enough to inhibit or prevent the one or more portions of the leaflets from moving into and/or through one or more openings in the frame during the crimping of the prosthetic valve, d) robust enough or strong enough to inhibit or prevent the one or more portions of the leaflets from tearing or pushing through the one or more layers of material and into and/or through one or more openings in the frame during the crimping of the prosthetic valve, e) strong enough or durable enough to resist damage during the crimp process of the frame of the prosthetic valve, and/or f) able to be nearly (e.g., 80-99.9999% and all values and ranges therebetween) or completely removable from the prosthetic valve after completion of the crimping of the prosthetic valve.

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve to inhibit or prevent damage to the one or more leaflets during the crimping of the prosthetic valve, and wherein the temporary/removeable barrier material is partially or fully formed of a flexible polymer. In one non-limiting embodiment, the flexible polymer that is used to partially or fully form the temporary/removeable barrier material is a partially or fully non-sintered PTFE material (i.e., a non-sintered PTFE material is PTFE material that has not been subjected to temperatures greater than 250° C.-300° C. and all values and ranges therebetween). In another non-limiting configuration, the flexible polymer used to partially of fully form the temporary/removeable barrier material is a dry material (e.g., absent lubricants, etc.). The temporary/removeable barrier material can be used alone or in conjunction with another device (e.g., compressible sleeve, compressible foam sleeve, a leaflet folding device, etc.) to facilitate in inhibiting or preventing damage to one or more leaflets during the crimping of the frame. The flexible polymer that is used to partially or fully form the temporary/removeable barrier material can optionally be colored (e.g., white, orange, red, yellow, pink, blue, etc.) to facilitate in the ability of the user to verify the removal of a portion or all of the flexible polymer from the prosthetic valve after completion of the crimping of the prosthetic valve.

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve to inhibit or prevent damage to the one or more leaflets during the crimping of the prosthetic valve, and wherein one or more layers of the temporary/removeable barrier material is partially or fully formed of a flexible polymer in the formed of a tape or strip. The polymer tape or strip may or may not be an adhesive tape or strip. In one non-limiting embodiment, the polymer tape or strip of the temporary/removeable barrier material is not an adhesive tape or strip and does not include any type of adhesive. In another non-limiting embodiment, the width of the polymer tape or strip of the temporary/removeable barrier material is at least 0.1 inches (e.g., 0.1-4 inches and all values and ranges therebetween). In another non-limiting embodiment, the thickness of each layer of the polymer tape or strip of the temporary/removeable barrier material is at least 1 mill (0.001 inches) (e.g., 0.001-0.125 inches and all values and ranges therebetween). In another non-limiting embodiment, the polymer tape or strip of the temporary/removeable barrier material can be wrapped one or more times (e.g., 1-10 times and all values and ranges therebetween; 1.001-2 times all values and ranges therebetween; 1.001-1.6 times all values and ranges therebetween) about a portion or all of the frame of the prosthetic valve. During the insertion of the temporary/removeable barrier material about the frame of the prosthetic valve, the temporary/removeable barrier material may or may not be stretched or expanded (unexpanded or expanded (single direction expanded or multi-axially expanded)). In another non-limiting configuration, the polymer tape or strip can be wrapped one or more times about the frame of the prosthetic valve, and a portion of the tape or strip of the temporary/removeable barrier material can be left unwrapped from the frame to form a handle, flap, tab, tail or grasping region that can be used to facilitate in the removal of the polymer tape or strip of temporary/removeable barrier material from the prosthetic valve after completion of the crimping of the prosthetic valve. The length of the handle, flap, tab, tail or grasping region, when formed, is non-limiting (e.g., 0.05-4 inches and all values and ranges therebetween).

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve to inhibit or prevent damage to the one or more leaflets during the crimping of the prosthetic valve, and wherein one or more layers of the temporary/removeable barrier material is partially or fully formed of a flexible polymer, and wherein the flexible polymer is in the form of a sleeve that has a cavity that extends along 70-100% (and all values and ranges therebetween) a longitudinal length of the sleeve. In one non-limiting embodiment, the length of the sleeve is at least 0.25 inches (e.g., 0.25-5 inches and all values and ranges therebetween). In another non-limiting embodiment, the thickness of the wall of the sleeve is at least 1 mill (0.001 inches) (e.g., 0.001-0.5 inches and all values and ranges therebetween). The wall of the sleeve can be formed of one or more layers of flexible polymer. In one non-limiting configuration, the sleeve is formed of PTFE material that has a thickness of 0.001-0.25 inches. The sleeve can be formed by an extrusion process, formed from a strip of flexible polymer, or formed by some other process. When the sleeve is formed from a strip of flexible polymer, the sleeve can be formed by a) optionally expanding the strip in a uniaxial or multi-axial direction to add strength to the strip, b) wrapping a section of the strip around a tubular structure (e.g., metal tube, ceramic tube, etc.) with a some overlap of the strip, and c) optionally heating the overlapped portions of the strip (e.g., heat above 200° C.-310° C. and all values and ranges therebetween), and/or optionally applying pressure to the overlapped portions of the strip to create texturing and/or bonding together of the overlapped layers and/or optionally adhesively connect together the overlapping portions to facilitate in forming a bonding between the overlapping layers and/or a more robust wrapped structure. In one non-limiting configuration, when the overlapped portions of the strip of polymer material are bonded together and/or textured, such region can optionally be sintered due to the high bonding temperatures, and wherein 80-100% (and all values and ranges therebetween) of the non-overlapped portions of the sleeve are non-sintered. In another non-limiting configuration, the overlapped portions of the sleeve form no more than 25% (e.g., 0.001-25% and all values and ranges therebetween) of the total outer surface or outer perimeter of the sleeve along the longitudinal length of the sleeve.

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve to inhibit or prevent damage to the one or more leaflets during the crimping of the prosthetic valve, and wherein the leaflet protector is absent a compressible sleeve that includes or is formed of a foam layer and/or other type of compressible layer. As defined herein, a compressible layer of material is a layer of material that can be reduced in thickness by at least 25% (e.g., 25-95% and all values and ranges therebetween) without damaging the layer of material.

In another and/or alternative non-limiting aspect of the present disclosure, there is provided one non-limiting method for using the leaflet protector that is or includes one or more layers of a temporary/removable barrier material, and wherein one or more layers of the temporary/removeable barrier material are partially or fully formed of a flexible polymer, and includes the step of first wrapping the flexible polymer layer one or more times about an outer surface of at least a portion of the frame of the prosthetic valve (e.g., wrapped 1-10 times and all values and ranges therebetween, wrapped 1.001-2 times and all values and ranges therebetween, wrapped 1.001-1.6 times and all values and ranges therebetween, etc.). During the crimping process, the one or more layers of a temporary/removable barrier material are caused to be compressed and a portion of the one or more layers of temporary/removeable barrier material are caused to move into and/or through one or more openings in the frame of the prosthetic valve. As a portion of the one or more layers of a temporary/removable barrier material move into and/or through one or more openings in the frame of the prosthetic valve during the crimping of the frame, such movement one or more layers of a temporary/removable barrier material into and/or through one or more openings in the frame inhibits or prevents one or more portions of the leaflets to move into and/or through one or more openings in the frame of the prosthetic valve, thus inhibiting or preventing damage to one or more or all of the leaflets from the crimping of the frame during the crimping of the prosthetic valve.

In another and/or alternative non-limiting aspect of the present disclosure, there is provided one non-limiting method for using the leaflet protector that is or includes one or more layers of a temporary/removeable barrier material, and includes the steps of a) wrapping the temporary/removeable barrier material one or more times about an outer surface of at least a portion of the frame of the prosthetic valve, b) crimping the prosthetic valve, and c) removing the temporary/removeable barrier material from the prosthetic valve. The prosthetic valve can optionally be further crimped after i) the temporary/removeable barrier material is removed from the prosthetic valve, ii) the compressible sleeve, when used, is removed from the prosthetic valve, but the temporary/removeable barrier material remains on the prosthetic valve for further crimping of the prosthetic valve after the compressible sleeve is removed, or iii) both the temporary/removeable barrier material and the compressible sleeve, when used, are removed from the prosthetic valve prior to further crimping of the prosthetic valve.

In another and/or alternative non-limiting aspect of the present disclosure, there is provided one non-limiting method for using the leaflet protector that is or includes one or more layers of a temporary/removeable barrier material, and includes the steps of a) wrapping the temporary/removeable barrier material one or more times about an outer surface of at least a portion of the frame of the prosthetic valve, b) partially crimping the prosthetic valve while the prosthetic valve is positioned about the shaft of the delivery system, but not about the inflatable balloon on the delivery system, c) sliding or otherwise inserting the prosthetic valve onto at least a portion of the inflatable balloon on the delivery system after the prosthetic valve has been partially crimped, d) further crimping the prosthetic valve on the inflatable balloon, and e) removing the temporary/removeable barrier material after final crimping of the prosthetic valve. The one or more layers of temporary/removeable barrier material can be removed from the prosthetic valve 1) after the prosthetic valve has been crimped and then inserted into the shaft portion of the delivery system (if the initial crimping of the prosthetic valve did not occur while the prosthetic valve was positioned about the shaft), 2) after the prosthetic valve has been inserted into the shaft portion of the delivery system and after the prosthetic valve has been partially or fully crimped on the shaft portion of the delivery system, 3) prior to or after the prosthetic valve has been inserted about a portion or all of the inflatable balloon of the delivery system, or 4) after the prosthetic valve has been inserted about a portion or all of the inflatable balloon of the delivery system and after the prosthetic valve has been crimped while inserted about a portion or all of the inflatable balloon of the delivery system. So long as the one or more layers of temporary/removeable barrier material remain on the frame during a portion or the complete crimping process, the portion of the frame that is covered by the one or more layers of temporary/removeable barrier material during the crimping process inhibits or prevents one or more portions of one or more leaflets from partially or fully passing through opening in the frame during a crimping process, thereby inhibiting or preventing damage to the leaflets during the crimping process. Prior to the prosthetic valve being inserted into a patient, the one or more layers of temporary/removeable barrier material are removed from the prosthetic valve. When an optional handle, flap, tab, tail or grasping region is formed on the temporary/removeable barrier material, the optional a handle, flap, tab, tail or grasping region can be used to facilitate in the removal of the one or more layers of temporary/removeable barrier material from the prosthetic valve.

In another and/or alternative non-limiting aspect of the present disclosure, there is provided a leaflet protector that is or includes one or more layers of a temporary/removeable barrier material that is partially or fully inserted or wrapped about a portion or all of the outer surface of the frame of the prosthetic valve prior to the partial or full crimping of the frame of the prosthetic valve to inhibit or prevent damage to the one or more leaflets during the crimping of the prosthetic valve, and wherein one or more layers of temporary/removeable barrier material are optionally used in conjunction with a compressible sleeve to facilitate in inhibiting or preventing damage to one or more leaflets during the crimping of the frame. When the leaflet protector includes a compressible sleeve and one or more layers of a temporary/removeable barrier material, one non-limiting method for using the leaflet protector includes a) the temporary/removeable barrier material is first wrapped one or more times about an outer surface of at least a portion of the frame of the prosthetic valve prior to the partial of full crimping of the frame, and b) thereafter the compressible sleeve is inserted about a portion or all of the frame of the prosthetic valve and about a portion or all of the temporary/removeable barrier material prior the partial or full crimping of the frame. During the crimping process, the compressible sleeve is caused to be compressed and a portion of the compressible material of the compressible sleeve a) optionally moves into and/or through one or more openings in the frame of the prosthetic valve, thereby causing a portion of the temporary/removeable barrier material to also be moved into and/or through one or more openings in the frame of the prosthetic valve, or b) causing a portion of the temporary/removeable barrier material to also be moved into and/or through one or more openings in the frame of the prosthetic valve, and the compressible material does not moved into and/or through one or more openings in the frame of the prosthetic valve. When a) both a portion of the compressible material of the compressible sleeve and a portion of the temporary/removeable barrier material moves into and/or through one or more openings in the frame of the prosthetic valve, or b) a portion of the compressible material of the compressible sleeve causes a portion of the temporary/removeable barrier material to move into and/or through one or more openings in the frame of the prosthetic valve while the compressible material does not moved into and/or through one or more openings in the frame of the prosthetic valve; such movement of both a portion of the compressible material of the compressible sleeve and a portion of the temporary/removeable barrier material, or only a portion of the temporary/removeable barrier material into one or more openings of the frame inhibits or prevents one or more portions of the one or more leaflets to move into and/or through one or more openings in the frame of the prosthetic valve, thus inhibiting or preventing damage to one or more or all of the leaflets from the crimping of the frame during the crimping of the prosthetic valve.

In another and/or alternative non-limiting aspect of the present disclosure, the is provided one non-limiting method for using the leaflet protector when crimping a prosthetic valve that includes a) wrapping the temporary/removeable barrier material one or more times about an outer surface of at least a portion of the frame of the prosthetic valve, b) inserting a compressible sleeve about a portion or all of the frame of the prosthetic valve and about a portion or all of the temporary/removeable barrier material, c) crimping the prosthetic valve, and d) removing the compressible sleeve and the temporary/removeable barrier material from the prosthetic valve. The prosthetic valve can optionally be further crimped after the compressible sleeve and the temporary/removeable barrier material are removed from the prosthetic valve. As can be appreciated, prosthetic valve can optionally be further crimped after the compressible sleeve is removed from the prosthetic valve, but while the temporary/removeable barrier material remains on the frame of the prosthetic valve.

In another and/or alternative non-limiting aspect of the present disclosure, there is provided one non-limiting method for using the leaflet protector when crimping a prosthetic valve that includes a) wrapping the temporary/removeable barrier material one or more times about an outer surface of at least a portion of the frame of the prosthetic valve, b) inserting a compressible sleeve about a portion or all of the frame of the prosthetic valve and about a portion or all of the temporary/removeable barrier material, c) partially crimping the prosthetic valve, d) removing the compressible sleeve from the prosthetic valve, e) further crimping the prosthetic valve while the temporary/removeable barrier material remains partially or fully about the frame of the prosthetic valve, and f) removing the temporary/removeable barrier material from the prosthetic valve after the further crimping of the prosthetic valve. The prosthetic valve can be optionally removed from the crimper prior to the removal of the compressible sleeve and/or the temporary/removeable barrier material from the prosthetic valve.

In another and/or alternative non-limiting aspect of the present disclosure, there is provided one non-limiting method for using the leaflet protector when crimping the prosthetic valve that includes a) wrapping the temporary/removeable barrier material one or more times about an outer surface of at least a portion of the frame of the prosthetic valve, b) inserting a compressible sleeve about a portion or all of the frame of the prosthetic valve and about a portion or all of the temporary/removeable barrier material, c) partially crimping the prosthetic valve, d) removing the compressible sleeve from the prosthetic valve, and wherein the prosthetic valve is optionally removed from the crimper prior to the removal of the compressible material from the prosthetic valve, e) optionally further crimping the prosthetic valve while the temporary/removeable barrier material remains partially or fully about the frame of the prosthetic valve, f) optionally inserting the crimped prosthetic valve onto the shaft portion of the delivery system (if the initial crimping of the prosthetic valve did not occur while the prosthetic valve was positioned about the shaft), and thereafter optionally further crimping the prosthetic valve on the shaft, g) optionally sliding or otherwise inserting the prosthetic valve onto at least a portion of an optional inflatable balloon, and thereafter optionally further crimping of the prosthetic valve on the optional inflatable balloon. The one or more layers of temporary/removeable barrier material can be removed from the prosthetic valve 1) after the compressible sleeve is removed from the prosthetic valve and prior to the prosthetic valve being further crimped, 2) after the compressible sleeve is removed from the prosthetic valve, and after the prosthetic valve has been further crimped after removal of the compressible sleeve, but prior to the prosthetic valve being inserted into the shaft portion of the delivery system (if the initial crimping of the prosthetic valve did not occur while the prosthetic valve was positioned about the shaft), 3) after the prosthetic valve has been inserted into the shaft portion of the delivery system (if the initial crimping of the prosthetic valve did not occur while the prosthetic valve was positioned about the shaft), but prior to any further crimping of the frame, 4) after the prosthetic valve has been inserted into the shaft portion of the delivery system and after the prosthetic valve has been crimped on the shaft portion of the delivery system, but prior to any further crimping of the frame, 5) after the prosthetic valve has been inserted about a portion or all of the inflatable balloon of the delivery system, but prior to any further crimping of the frame, or 6) after the prosthetic valve has been inserted about a portion or all of the inflatable balloon of the delivery system and after the prosthetic valve has been crimped while inserted about a portion or all of the inflatable balloon of the delivery system. So long as the one or more layers of temporary/removeable barrier material remain on the frame during a crimping process, the portion of the frame that is covered by the one or more layers of temporary/removeable barrier material inhibits or prevents one or more portions or one or more leaflets from partially or fully passing through opening in the frame during a crimping process, thereby inhibiting or preventing damage to the leaflets during the crimping process. Prior to the prosthetic valve being inserted into a patient, the one or more layers of temporary/removeable barrier material are removed from the prosthetic valve. When a handle, flap, tab, tail or grasping region is formed on a portion of the temporary/removeable barrier material, the a handle, flap, tab, tail or grasping region can be used to facilitate in the removal of the one or more layers of temporary/removeable barrier material from the prosthetic valve.

In another and/or alternative non-limiting aspect of the present disclosure, there is provided a leaflet protector that includes one or more layers of a temporary/removeable barrier material that are partially or fully inserted into a cavity of a compressible sleeve, and thereafter the compressible sleeve and temporary/removeable barrier material are partially or fully positioned about the outer surface of the frame of the prosthetic valve prior to the full or partial crimping of the frame, and wherein the temporary/removeable barrier material is used in conjunction with the compressible sleeve to facilitate in inhibiting or preventing damage to one or more leaflets during the crimping of the prosthetic valve. The one or more layers of the temporary/removeable barrier material in the cavity of a compressible sleeve can optionally be adhesive connected, melt connected, stitched, mechanically connected, clamped, and/or connected by other arrangement to the compressible sleeve. As can be appreciated, the one or more layers of the temporary/removeable barrier material in the cavity of a compressible sleeve can optionally be detachably positioned in the cavity of the compressible sleeve so that the compressible sleeve can be optionally removed from the prosthetic valve while the temporary/removeable barrier material remains on a portion or all of the outer surface of the frame for further crimping of the prosthetic valve. In another non-limiting embodiment, the temporary/removeable barrier material can optionally include one or more handles, flaps, tabs, tails or grasping regions that can be used to facilitate in the removal of the temporary/removeable barrier material from the prosthetic valve. In one non-limiting configuration, the one or more handles, flaps, tabs, tails or grasping regions extend outwardly from one or both ends of the cavity of the compressible sleeve, and can be optionally folded over a portion of the outer surface of the compressible sleeve to facilitate in holding a portion of the temporary/removeable barrier material within the cavity of the compressible sleeve during the insertion of the compressible sleeve and temporary/removeable barrier material about a portion of the prosthetic valve. The one or more handles, flaps, tabs, tails or grasping regions can also be configured (e.g., bent, folded, held, etc.) to enable the compressible sleeve to be removed from the prosthetic valve while the temporary/removeable barrier material remains about a portion or all of the frame of the prosthetic valve.

In another and/or alternative non-limiting aspect of the present disclosure, there is provided a leaflet protector that includes one or more layers of a temporary/removeable barrier material that are performed in a particular shape to facilitate in the proper insertion and use of the temporary/removeable barrier material on the prosthetic valve. The use of a polymer tape (e.g., PTFE tape, etc.) as a portion or all of the temporary/removeable barrier material to inhibit or prevent protrusion of one or more portions of one or more leaflets into one or more openings of a frame during crimping of the prosthetic valve can be challenging for many users since the polymer tape can be difficult to manipulate about the frame of the prosthetic valve, especially when the user is wearing gloves. The polymer tape generally a) must be applied over a certain portion of the prosthetic valve and not on other portions of the prosthetic valve, b) must remain in place during handling of the prosthetic valve and the crimping of the prosthetic valve, and/or c) should be easily removable after the crimping of the prosthetic valve. Such tasks can be difficult for a user wearing gloves. In one non-limiting embodiment, there is provided a temporary barrier assembly that is formed of one or more layers of polymer tape that is preformed into a particular shape so as to improve the usability of the temporary barrier assembly with a prosthetic valve and to ensure that the proper regions of the prosthetic valve are covered by the temporary barrier assembly prior to the crimping of the prosthetic valve. In one non-limiting configuration, the temporary barrier assembly is formed of one or more layers of non-sintered PTFE tape, and wherein the temporary barrier assembly is formed into a preformed configuration by the following steps of: a) cutting a length of PTFE tape that is greater than the circumference of the prosthetic valve that is in the expanded orientation (e.g., 1.1 times to 5 times the circumference of the prosthetic valve that is in the expanded orientation [i.e., precrimped orientation] and all values and ranges therebetween), b) optionally cutting a portion of the longitudinal length of PTFE tape to create a tab or tail at least one of the end regions of the PTFE tape to be used as a removal tail or tab for later removal of the PTFE tape from the prosthetic valve after the crimping of the prosthetic valve, c) winding the PTFE tape about at least a portion of a mandrel (e.g., circular mandrel, etc.), which mandrel has a cross-sectional area or diameter that is equal to or greater than that of the uncrimped prosthetic valve (e.g., 1-1.5 times the cross-sectional area or diameter of the uncrimped prosthetic valve and all values and ranges therebetween), and wherein a portion of the wrapped PTFE tape extends off the front end of the mandrel (e.g., 1-20% of a longitudinal length of the tube of wrapped PTFE extends forwardly of the front end of the mandrel and all values and ranges therebetween), and wherein the portion of the tube of wrapped PTFE tape that is located rearwardly of the front end of the mandrel (i.e., portion of the PTFE tape that is wound about the mandrel) is the desired length of the PTFE tape that is to be positioned about the prosthetic valve prior to the crimping of the prosthetic valve, d) optionally rolling a textured surface against the outer surface of the PTFE tape that is wrapped about the mandrel to increase the mechanical adherence of overlapping portions of the PTFE on the mandrel so as to improve the robustness and integrity of the tube of wrapped PTFE tape when the tube of PTFE tape is removed from the mandrel, e) pulling a portion of PTFE tape that extends forwardly of the front end of the mandrel away from the front end of the mandrel in a direction that is parallel or close to parallel to a longitudinal axis of the mandrel so as to stretch the PTFE tape in a direction that perpendicular or near perpendicular (0-20° to perpendicular and all values and ranges therebetween) to the direction of extrusion of the PTFE tape, thereby resulting in the PTFE tape stretching in such direction, and wherein the front end of the mandrel constricts the stretching of the PTFE tape that is positioned rearward of the front end of the mandrel so as to create a wrapped PTFE tape assembly that has a front portion that has an inner cavity that has a smaller diameter or cross-sectional area than the diameter or cross-sectional area of the rear portion of the PTFE tape assembly that is located on the mandrel, and wherein if the PTFE tape assembly includes a flap or tab, then the flap or tab is positioned at the front end portion of the PTFE tape assembly that extends off the front end of the mandrel, f) removing the PTFE assembly from the mandrel, and wherein the PTFE tape has a front portion that has an inner cavity that has a smaller diameter or cross-sectional area than the inner cavity of the rear portion of the wrapped PTFE tape that was formally wrapped about the mandrel so as to create a “hat shaped” PTFE tape assembly, g) positioning the rear portion of “hat” shaped PTFE tape assembly over a portion of the prosthetic valve, and wherein the smaller diameter or cross-sectional area front portion of the wrapped PTFE tape assembly prevents the front portion of the wrapped PTFE tape portion from being positioned over or moved over the prosthetic valve, thereby ensuring that the designed length of the rear portion of the wrapped PTFE tape assembly that has the larger cross-sectional area or diameter overlies the desired portion of the prosthetic valve during the crimping process. After the prosthetic valve is crimped, the PTFE tape assembly can be removed from the prosthetic valve by a user grasping the optional tap or flap on the PTFE tape assembly and then pulling the PTFE tape assembly off of the crimped prosthetic valve. Even when the PTFE tape assembly has no tab or flap, the PTFE tape assembly can still be removed from the crimped prosthetic valve.

In another and/or alternative non-limiting aspect of the present disclosure, there is provided a leaflet folding device that can optionally be used with the crimper device and/or the leaflet protector in accordance with the present disclosure to facilitate in obtaining a leaflet folding configuration during the crimping of the frame of a prosthetic valve so as to obtain a desired folded leaflet configuration after the frame has been fully crimped. In one non-limiting embodiment, the leaflet folding device is configured to bend or facilitate in the bending of a portion of one or more or all of the leaflets of the prosthetic valve prior to, and/or during a prior or all of the crimping process. Such bending of the one or more leaflets by the leaflet folding device occurs a) prior to the initial crimping of the frame of the prosthetic valve, and/or b) during the crimping of the frame of the prosthetic valve. In another non-limiting method of use, prior to and/or during the crimping of the frame, a portion or all of the leaflet folding device is rotated (e.g., 2-180° and all values and ranges therebetween) about the longitudinal axis of the frame (e.g., central longitudinal axis of the frame, etc.) so as to facilitate in the folding of the leaflets during the crimping of the frame. Such rotation of the is used to facilitate in the folding of the leaflets. Such rotation of the one or more leaflets by the leaflet folding device occurs a) prior to the initial crimping of the frame of the prosthetic valve, and/or b) during the crimping of the frame of the prosthetic valve. The leaflet folding device can optionally be removed from or disengaged from the one or more leaflets prior to the outflow end of the frame of the prosthetic valve being 5-99% fully crimped (and all values and ranges therebetween).

In another and/or alternative non-limiting aspect of the present disclosure, the medical device is a prosthetic valve (e.g., TAVR valve, prosthetic aortic valve, prosthetic mitral valve, prosthetic tricuspid valve, prosthetic pulmonary valve, prosthetic valve for used in left atrial appendage, etc.). In one non-limiting embodiment, the medical device includes an expandable frame, more particularly the medical device is in the form of a cardiovascular implant for the treatment of structural heart disease wherein the cardiovascular implant includes an expandable frame, and still more particularly to a medical device is in the form of a prosthetic valve for the treatment of structural heart disease wherein the prosthetic valve includes an expandable frame that is optionally formed of a rhenium and/or hafnium containing metal alloy; however, other metal alloys can be used. In one non-limiting embodiment, the frame has a novel geometry and/or the frame is partially (e.g., 10-99.99 wt. % and all values and ranges therebetween) or fully formed of a rhenium and/or hafnium containing alloy to enable the frame to have one or more of the following properties: a) an open cell geometry that can be used to reduce delivery system size thereby reducing vascular and neurological complications, b) an open cell geometry that has high radial strength due to the high yield strength and ultimate tensile strength of the rhenium and/or hafnium containing metal alloy, c) improved restoration of the physiologic EOA in challenging, heavily calcified valves that exert high force on the bioprosthetic valve, while also allowing a reduced crimp diameter for vascular access, d) improved restoration of the physiologic EOA that results in greater longevity of the bioprosthetic valve, e) lower recoil than traditional materials used to form frames such as stainless steel, chromium-cobalt, or titanium alloys, thereby resulting in less recoil of the frame when expanded which leads to decreased risk of valve embolization, decreased paravalvular leak due to improved conformability of the native anatomy, more accurate restoration of the physiologic EOA, and decrease conduction system injury due to a lower balloon inflation diameter required to obtain the physiologic EOA after balloon inflation, f) an open cell geometry that is configured to have little (e.g., 0-20% longitudinal foreshortening along a longitudinal axis of the expandable frame and all values and ranges therebetween) or no foreshortening when expanded, which allows for more accurate placement of the prosthetic valve at the treatment site, and wherein a frame that has little or no longitudinal foreshortening when expanded can be expanded with a shorter balloon, which use of a shorter balloon for frame expansion can decrease conduction system injury, g) commissural alignment markers and an open cell geometry between the commissures that allows for proper placement of the prosthetic valve in relation to the native commissures of the valve for proper hemodynamic function in regard to wash out of the prosthetic valve and blood flow to the coronaries, which leads to better durability and longevity of the prosthetic valve, and access and re-intervention of the coronaries preventing future adverse events, h) an open cell geometry with radial symmetry, longitudinal symmetry, and little or no longitudinal foreshortening which allows for symmetrical and cylindrical expansion of the prosthetic valve resulting in lower rates of leaflet thrombosis and structural valve deterioration, and/or i) the metal alloy contains no nickel content thereby preventing allergic response due to the presence of nickel and restenosis associated with nickel content.

In another and/or alternative non-limiting aspect of the present disclosure, the prosthetic valve includes a radially collapsible and expandable frame and a leaflet structure that comprises a plurality of leaflets, optionally an inner skirt, and optionally an outer skirt that is disposed on and partially or fully covering or overlaid over the cells of at least a portion of the frame.

In another and/or alternative non-limiting aspect of the present disclosure, at least a portion of the medical device is configured to be radially collapsible to a collapsed or crimped orientation for introduction into the body (e.g., on a delivery catheter, etc.) and radially expandable to an expanded orientation for implanting the medical device at a desired location in the body. The frame of the medical device can be formed of a plastically-expandable material that permits crimping of the frame to a smaller profile for delivery and expansion of the frame at the treatment site. The expansion of the crimped frame of the medical device can be by an expansion device such as, but not limited to, a balloon of on a balloon catheter.

In another and/or alternative non-limiting aspect of the present disclosure, there is provided a prosthetic valve that includes an inner skirt that is positioned at least partially about the interior region of the expandable frame, and wherein inner skirt can be formed of a variety of materials (e.g., polymer [e.g., polyethylene terephthalate (PET), polyester, nylon, Kevlar®, silicon, etc.], composite material, metal, fabric material, etc.). In one non-limiting embodiment, the material used to partially or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with the stretchable and/or compressible material to provide additional structural integrity to the inner skirt. In another non-limiting embodiment, the inner skirt can be made out of a woven material; however, non-woven materials can also or alternatively be used. The size, configuration, and thickness of the inner skirt is non-limiting. The thickness of the inner skirt is generally 0.1-20 mils (and all values and ranges therebetween). In one non-limiting configuration, the configuration of the inner skirt and outer skirt are optionally generally different (e.g., knit pattern is different, formed of different types of layers, formed of different number of layers, etc.), and can optionally be formed of different materials. In another non-limiting configuration, the inner skirt is optionally formed of a less flexible and/or compressible material than the optional outer skirt; however, this is not required. In another non-limiting configuration, the inner skirt is optionally formed of tighter knit pattern than the knit pattern of the outer skirt. In another non-limiting configuration, the inner skirt is optionally is formed of a thinner material than the material used to form the outer skirt. In another non-limiting configuration, the inner skirt is optionally formed of a material and/or knit pattern that forms a less compressible material with regard to material thickness compression than the material used to form the outer skirt. In one non-limiting configuration, the porosity of the inner skirt is optionally less than the porosity of the outer skirt (e.g., the porosity of the outer skirt is 1.01 to 100 times (and all values and ranges therebetween) the porosity of the inner skirt).

In another and/or alternative non-limiting aspect of the present disclosure, there is provided a prosthetic valve that includes an inner skirt that is positioned at least partially about the interior region of the expandable frame, and wherein inner skirt is connected to a) one or more leaflets, b) the frame, and/or c) to the outer skirt. The inner skirt can be connected to a) one or more leaflets, b) the frame, and/or c) to the outer skirt by a variety of arrangements (e.g., sutures, adhesive, melted connection, clamping arrangement, stitches, etc.).

In another and/or alternative non-limiting aspect of the present disclosure, there is provided a prosthetic valve that includes an outer skirt that is positioned at least partially about the outer or exterior region of the expandable frame. The outer skirt can be formed of a variety of materials (e.g., polymer [e.g., polyethylene terephthalate (PET), polyester, nylon, Kevlar®, silicon, etc.], composite material, metal, fabric material, etc.). In one non-limiting embodiment, the material used to partially or fully form the outer skirt can optionally be substantially non-elastic (i.e., substantially non-stretchable). In another non-limiting embodiment, the material used to partially or fully form the outer skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with a flexible material or with a stretchable and/or compressible material so as to provide additional structural integrity to the outer skirt.

In another and/or alternative non-limiting aspect of the present disclosure, there is provided a prosthetic valve that includes an outer skirt that is positioned at least partially about the outer or exterior region of the expandable frame, wherein the outer skirt is formed of a knitted polymeric construct. The knitted polymeric construct (e.g., knitted PET, etc.) provides for a large volume outer skirt, a highly compressible outer skirt, and/or an outer skirt having known biocompatibility. In one non-limiting embodiment, the knitted polymeric construct used to partially or fully form the outer skirt creates a large surface area and/or volume that is used to partially or fully close, block, seal, etc. gaps that exist between the expanded prosthetic valve (e.g., prosthetic valve, etc.) at a treatment site and the surrounding tissue at the treatment site so as to reduce or prevent leakage (e.g., paravalvular leakage, etc.) about the prosthetic valve when expanded and inserted at the treatment site. In another non-limiting embodiment, the knitted polymeric construct used to partially or fully form the outer skirt also has a large degree of compressibility a) so that the prosthetic valve can be crimped on a delivery device and maintain a small crimped profile, and/or b) so it can be compressed during i) the crimping of the valve on a delivery device, and/or ii) during the insertion of the prosthetic valve through an introducer sheath.

In another and/or alternative non-limiting aspect of the present disclosure, there is provided a prosthetic valve that includes an outer skirt that is positioned at least partially about the outer or exterior region of the expandable frame, wherein the outer skirt is formed of a knitted polymeric construct that has specially engineered loops and loop lengths that are configured to protrude from the knitted polymeric construct. In one non-limiting embodiment, the engineered loops are engineered to enable a low compressed profile of the crimped valve that includes the outer skirt, and/or to create a maximize volume about the expanded or deployed prosthetic valve at a treatment site so as to partially (e.g., fill 50-99.99% and all values and ranges therebetween) or fully fill any space between the deployed prosthetic valve and the patient's native aortic annulus or other cardiovascular site or vascular site that the prosthetic valve is deployed (e.g., minimize or prevent paravalvular leakage, etc.). In another non-limiting embodiment, the knitted polymeric construct can optionally be used to partially or fully form the outer skirt can be configured to promote localized clotting about the deployed prosthetic valve at the treatment site to cause further sealing about the prosthetic valve after deployment at the treatment site (e.g., minimize or prevent paravalvular leakage, etc.).

In another and/or alternative non-limiting aspect of the present disclosure, there is provided a prosthetic valve that includes an outer skirt that is positioned at least partially about the exterior region of the expandable frame, and wherein outer skirt is connected to a) one or more leaflets, b) the frame, and/or c) to the inner skirt. The outer skirt can be connected to a) one or more leaflets, b) the frame, and/or c) to the inner skirt by a variety of arrangements (e.g., sutures, adhesive, melted connection, clamping arrangement, stitches, etc.).

In another and/or alternative non-limiting aspect of the present disclosure, there is provided a prosthetic valve that includes one or more leaflets that are connected to a) the expandable frame, b) the optional inner skirt, and/or c) the optional outer skirt. In one non-limiting embodiment, there is provided a prosthetic valve that includes a leaflet arrangement that can be attached to the frame and/or the inner and/or outer skirt. The connection arrangement used to secure the leaflet arrangements to the frame and/or inner and/or outer skirt is non-limiting (e.g., sutures, staples, melted bold, adhesive, stitches, clamp arrangement, etc.).

In another and/or alternative non-limiting aspect of the present disclosure, the material used to form the leaflets of the leaflet arrangement can include polymers, bovine pericardial tissue, bovine tissue, porcine tissue, biocompatible synthetic materials, or various other suitable natural or synthetic materials. The material and/or tissue used to form the one or more leaflets can optionally be treated/stabilized through a method of collagen cross linking and thereafter stored dry or wet (e.g., tissue is stored dry after a glycerin-based dehydration process, etc.).

In another and/or alternative non-limiting aspect of the present disclosure, there is provided a prosthetic valve that includes a leaflet arrangement comprised of two or more leaflets (e.g., 2, 3, 4, 5, 6, etc.). In one non-limiting arrangement, the leaflet arrangement includes three leaflets arranged in a tricuspid arrangement.

In another and/or alternative non-limiting aspect of the present disclosure, the medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. One or more polymers optionally can be used to at least partially control the release of one or more agents from the medical device.

In another and/or alternative non-limiting aspect of the present disclosure, one or more surfaces of the medical device can optionally be treated to achieve the desired coating properties of the one or more agents and one or more polymers coated on the medical device. Such surface treatment techniques include, but are not limited to, cleaning, buffing, smoothing, nitriding, annealing, swaging, cold working, etching (chemical etching, plasma etching, etc.), etc.

In another and/or alternative non-limiting aspect of the present disclosure, the medical device can optionally include a marker material that facilitates enabling the medical device to be properly positioned in a body passageway (e.g., blood vessel, heart valve, etc.). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.).

In another and/or alternative non-limiting aspect of the present disclosure, the medical device can optionally be an expandable device that can be expanded by use of some other device (e.g., balloon, etc.).

In another and/or alternative non-limiting aspect of the present disclosure, the outer surface of the medical device is at least partially (e.g. 1-99.999 wt. % and all values and ranges therebetween) or fully coated with an enhancement coating. As defined herein, the enhancement coat is not an agent, a polymer layer that is used with an agent, or a nitride coating or layer. The enhancement coating is used to a) reduced metal ion release of the metal material from the medical device, b) reduce the rate of corrosion on the metal that forms the medical device, c) form an outer surface of the medical device that is absent nickel and/or chromium, d) increase a surface hardness of the medical device, e) promote the formation and/or release of nitric oxide (NO) from the implanted medical device, f) promote bone healing about the medical device, g) inhibit or reduce neointimal hyperplasia/cell overgrowth onto one or more portions of the medical device after implantation in the treatment area, h) reduce infection about the medical device after implantation in the treatment area, i) reduce bacterial growth and/or bacterial infection about the implanted medical device, j) improve the biocompatibility of the implanted medical device, k) reduce the incidence of nickel exposure and/or ion release from the medical device that can react with cells about the medical device after implantation in the treatment area, and/or l) reduce inflammatory cell response about the medical device after implantation in the treatment area. In another non-limiting embodiment, the enhancement coating on one or more portions of the medical device is formulated to provide and/or promote generation of nitric oxide near, at and/or in adjacent tissue after the prosthetic valve has been expanded at the treatment site. Nitric oxide can reduce neointimal hyperplasia, reduce tissue proliferation, reduce platelet activation, reduce thrombosis, reduce restenosis, and can promote endothelial cell angiogenesis, reduce coagulation, reduce inflammation, reduce the rate of smooth muscle growth and pannus formation, all of which can contribute to an improved pro-healing environment. In another non-limiting embodiment, the enhancement coating provides, promotes and/or facilitates in a) formation or generation of nitric oxide (NO), b) stimulation of cells about the implanted medical device, and/or c) enhance modulation of cells about the implanted medical device. In one non-limiting arrangement, the enhancement coating is a metal oxynitride layer that is deposited a portion (e.g., 1-99.990% of the outer surface and all values and ranges therebetween) or all of outer surface of the medical device. In one non-limiting specific configuration, the metal oxynitride layer is or includes titanium oxynitride and/or zirconium oxynitride. In another non-limiting specific configuration, thickness of the metal oxynitride layer is 10 nanometers to 10 microns (and all values and ranges therebetween). In one non-limiting specific configuration, the oxygen to nitrogen atomic ratios of the metal oxynitride layer is 1:10 to 10:1 (and all values and ranges therebetween). In another non-limiting specific configuration, the coating of metal oxynitride layer is optionally deposited onto a metallic adhesion layer in between the base substrate of the medical device and the oxynitride layer. In another non-limiting specific configuration, the adhesion layer, when used, optionally is or includes titanium metal and/or zirconium metal. The thickness of the adhesion layer optionally has a thickness of at least 10 nanometers (e.g., 10 to 500 nanometers and all values and ranges therebetween). When the metal oxynitride layer is deposited on a portion or all of the outer surface of the medical device, the metal oxynitride coating can be used to enhance healing about the medical device and/or to increase the operational life of the medical device. In one non-limiting configuration, the enhancement coating can be coated on a portion or all of a) the frame, b) the leaflet arrangement, c) the optional inner skirt, and/or d) the optional outer skirt.

One non-limiting object of the present disclosure is the provision of a device and method for crimping a crimpable or plastically deformable portion of a medical device.

Another and/or alternative non-limiting object of the present disclosure is the provision of a device and method for crimping a crimpable or plastically deformable portion of a medical device that includes one or more leaflets.

Another and/or alternative non-limiting object of the present disclosure is the provision of a device and method for crimping of the frame of the prosthetic valve, while reducing the risk of damage to the leaflets and/or other components of the prosthetic valve during the crimping process.

Another and/or alternative non-limiting object of the present disclosure is the provision of a device and method for crimping of the frame of the prosthetic valve that includes the use of a leaflet protector.

Another and/or alternative non-limiting object of the present disclosure is the provision of a device and method for crimping of the frame of the prosthetic valve that includes the use of a leaflet protector that is or includes one or more layers of a temporary/removeable barrier material that is partially or fully wrapped about a portion or all of the outer surface of the frame of the prosthetic valve prior to the crimping of the frame or prior to the full crimping of the frame of the prosthetic valve.

Another and/or alternative non-limiting object of the present disclosure is the provision of a device and method for crimping of the frame of the prosthetic valve that includes the use of a leaflet protector that is or includes one or more layers of a temporary/removeable barrier material is partially or fully wrapped about a portion or all of the outer surface of the frame of the prosthetic valve prior to the crimping of the frame or prior to the full crimping of the frame of the prosthetic valve, and wherein the one or more layers of a temporary/removeable barrier material are used to inhibit or prevent damage to one or more leaflets during the crimping of the frame.

Another and/or alternative non-limiting object of the present disclosure is the provision of a device and method for crimping of the frame of the prosthetic valve that includes the use of a leaflet protector wherein one or more layers of a temporary/removeable barrier material is formed of a material that a) is flexible so that the material can be partially or forced into the frame openings to inhibit or prevent one or more portions of the leaflets from moving into and/or through one or more openings in the frame, b) is thin enough so that the one or more layers of material can be partially or fully forced into the frame openings, c) is robust enough or strong enough to inhibit or prevent the one or more portions of the leaflets from moving into and/or through one or more openings in the frame, d) is robust enough or strong enough to inhibit or prevent the one or more portions of the leaflets from tearing through the one or more layers of material and into and/or through one or more openings in the frame, e) is strong enough or durable enough to resist damage during the crimp process of the frame of the prosthetic valve, and/or f) is able to be nearly (e.g., 80-99.9999% and all values and ranges therebetween) or completely removable from the prosthetic valve after that partial or full completion of the crimping of the prosthetic valve.

Another and/or alternative non-limiting object of the present disclosure is the provision of a device and method for crimping of the frame of the prosthetic valve that includes the use of a leaflet protector that includes one or more layers of a temporary/removeable barrier material formed of a material that fully or partially includes non-sintered PTFE material.

Another and/or alternative non-limiting object of the present disclosure is the provision of a device and method for crimping of the frame of the prosthetic valve that includes the use of a leaflet protector that includes one or more layers of a temporary/removeable barrier material formed of a material that includes a flexible polymer that is a dry material (e.g., absent lubricants, etc.).

Another and/or alternative non-limiting object of the present disclosure is the provision of a device and method for crimping of the frame of the prosthetic valve that includes the use of a leaflet protector that includes one or more layers of a temporary/removeable barrier material wherein the layer of temporary barrier can be wrapped one or more times (e.g., 1-10 times and all values and ranges therebetween) about the frame of the prosthetic valve.

Another and/or alternative non-limiting object of the present disclosure is the provision of a device and method for crimping of the frame of the prosthetic valve that includes the use of a leaflet protector that includes one or more layers of a temporary/removeable barrier material wherein a portion of the one or more layers of temporary/removeable barrier material can be left unwrapped from the frame to form a handle, flap, tab, tail or grasping region that can be used to facilitate in the removal of the temporary/removeable barrier material from the prosthetic valve after that partial or full completion of the crimping of the prosthetic valve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and which one or more layers of temporary/removeable barrier material are used in conjunction with the compressible sleeve to facilitate in inhibiting or preventing damage to one or more leaflets during the crimping of the frame.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein the one or more layers of the temporary/removeable barrier material are partially or fully wrapped about a portion or all of the outer surface of the frame of the prosthetic valve prior to the full or partial crimping of the frame of the prosthetic valve, and wherein the compressible sleeve is a) positioned about a portion or all of the one or more layers of the temporary/removeable barrier material are, and b) partially or fully positioned about the outer perimeter of the frame.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein a method for crimping a prosthetic valve includes a) wrapping the temporary/removeable barrier material one or more times about an outer surface of at least a portion of the frame of the prosthetic valve, b) inserting the compressible sleeve about a portion or all of the frame of the prosthetic valve and about a portion or all of the temporary/removeable barrier material, c) crimping the prosthetic valve, and d) removing the compressible sleeve and optional removing the temporary/removeable barrier material from the prosthetic valve; and wherein the prosthetic valve can optionally be further crimped after the compressible sleeve and/or the temporary/removeable barrier material are removed from the prosthetic valve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein a method for crimping a heart valve includes a) wrapping the temporary/removeable barrier material one or more times about an outer surface of at least a portion of the frame of the prosthetic valve, b) inserting the compressible sleeve about a portion or all of the frame of the prosthetic valve and about a portion or all of the temporary/removeable barrier material, c) partially crimping the prosthetic valve, d) removing the compressible sleeve from the prosthetic valve, e) further crimping the prosthetic valve while the temporary/removeable barrier material remains partially or fully about the frame of the prosthetic valve, and f) removing the temporary/removeable barrier material from the prosthetic valve after the further crimping of the prosthetic valve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein a method for crimping a heart valve includes a) wrapping the temporary/removeable barrier material one or more times about an outer surface of at least a portion of the frame of the prosthetic valve, b) inserting the compressible sleeve about a portion or all of the frame of the prosthetic valve and about a portion or all of the temporary/removeable barrier material, c) partially crimping the prosthetic valve, d) removing the compressible sleeve from the prosthetic valve, e) optionally further crimping the prosthetic valve while the temporary/removeable barrier material remains partially or fully about the frame of the prosthetic valve, f) optionally inserting the crimped prosthetic valve onto the shaft portion of the delivery system (if the initial crimping of the prosthetic valve did not occur while the prosthetic valve was positioned about the delivery system), and thereafter optionally further crimping the prosthetic valve on the shaft, g) optionally sliding or otherwise inserting the prosthetic valve onto at least a portion of an optional inflatable balloon, and thereafter optionally further crimping of the prosthetic valve on the optional inflatable balloon.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein the one or more layers of temporary/removeable barrier material can be removed from the prosthetic valve 1) after the compressible sleeve is removed from the prosthetic valve and prior to the prosthetic valve being further crimped, 2) after the compressible sleeve is removed from the prosthetic valve, and after the prosthetic valve has been further crimped after removal of the compressible sleeve, but prior to the prosthetic valve being inserted into the shaft portion of the delivery system (if the initial crimping of the prosthetic valve did not occur while the prosthetic valve was positioned about the shaft), 3) after the prosthetic valve has been inserted into the shaft portion of the delivery system, but prior to further crimping of the frame, 4) after the prosthetic valve has been inserted into the shaft portion of the delivery system and after the prosthetic valve has been crimped on the shaft portion of the delivery system, but prior to the prosthetic valve being placed on the inflatable balloon of the delivery device, 5) after the prosthetic valve has been inserted about a portion or all of the inflatable balloon of the delivery system, but prior to further crimping of the frame, or 6) after the prosthetic valve has been inserted about a portion or all of the inflatable balloon of the delivery system and after the prosthetic valve has been crimped while inserted about a portion or all of the inflatable balloon of the delivery system.

Another and/or alternative non-limiting object of the present disclosure is the provision of a device and method for crimping of the frame of the prosthetic that valve that includes the use of a leaflet protector that includes a compressible sleeve and/or one or more layers of a temporary/removeable barrier material, and wherein prior to the prosthetic valve being inserted into a patient, a) the one or more layers of temporary/removeable barrier material are removed from the prosthetic valve, and b) the compressible sleeve, if used, is removed from the prosthetic valve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein one or more layers of a temporary/removeable barrier material are partially or fully inserted into a cavity of a compressible sleeve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein one or more layers of a temporary/removeable barrier material are partially or fully inserted into a cavity of a compressible sleeve, and thereafter the compressible sleeve and temporary/removeable barrier material are partially or fully positioned about the outer surface of the frame of the prosthetic valve prior to the full or partial crimping of the frame, and which the temporary/removeable barrier material is used in conjunction with a compressible sleeve to facilitate in inhibiting or preventing damage to one or more leaflets during the crimping of the prosthetic valve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein one or more layers of a temporary/removeable barrier material are partially or fully inserted into a cavity of a compressible sleeve, wherein one or more layers of the temporary/removeable barrier material in the cavity of a compressible sleeve can optionally be adhesive connected, melt connected, stitched, and/or connected by other arrangement to the compressible sleeve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein one or more layers of a temporary/removeable barrier material are partially or fully inserted into a cavity of a compressible sleeve, and wherein the one or more layers of the temporary/removeable barrier material in the cavity of a compressible sleeve can optionally be detachably positioned in the cavity of the compressible sleeve so that the compressible sleeve can be optionally removed from the prosthetic valve while the temporary/removeable barrier material remains on a portion or all of the outer surface of the frame for further crimping of the prosthetic valve.

Another and/or alternative non-limiting object of the present disclosure is the provision of a device and method for crimping of the frame of the prosthetic valve that includes the use of a leaflet protector that includes a compressible sleeve and one or more layers of a temporary/removeable barrier material, and wherein one or more layers of a temporary/removeable barrier material are partially or fully inserted into a cavity of a compressible sleeve, and wherein the temporary/removeable barrier material can optionally include one or more handles, flaps, tabs, tails or grasping regions that can be used to a) facilitate in the removal of the temporary/removeable barrier material from the prosthetic valve, b) facilitate in holding a portion of the temporary/removeable barrier material within the cavity of the compressible sleeve during the insertion of the compressible sleeve and temporary/removeable barrier material about a portion of the prosthetic valve, and/or c) allow the compressible sleeve to be removed from the prosthetic valve while the temporary/removeable barrier material remains about a portion or all of the prosthetic valve.

Another and/or alternative non-limiting object of the present disclosure is the provision of a device and method for crimping of the frame of the prosthetic valve includes the use of a leaflet protector that includes one or more layers of a temporary/removeable barrier material that are performed in a particular shape to facilitate in the proper insertion and use of the temporary/removeable barrier material on the prosthetic valve.

Another and/or alternative non-limiting object of the present disclosure is the provision of a device wherein the temporary barrier assembly is formed of one or more layers of non-sintered PTFE tape, and wherein the temporary barrier assembly is formed into a formed configuration by the following steps: of a) cutting a length of PTFE tape that is greater than the circumference of the prosthetic valve that is in the expanded orientation (e.g., 1.1 times to 5 times the circumference of the prosthetic valve that is in the expanded orientation [i.e., precrimped orientation] and all values and ranges therebetween), b) optionally cutting a portion of the longitudinal length of PTFE tape to create a tab or tail at least one of the end regions of the PTFE tape to be used as a removal tail or tab for later removal of the PTFE tape from the prosthetic valve after the crimping of the prosthetic valve, c) winding the PTFE tape about at least a portion of a mandrel (e.g., circular mandrel, etc.), which mandrel has a cross-sectional area or diameter that is equal to or greater than that of the uncrimped prosthetic valve (e.g., 1-1.5 times the cross-sectional area or diameter of the uncrimped prosthetic valve and all values and ranges therebetween), and wherein a portion of the wrapped PTFE tape extends off the front end of the mandrel (e.g., 1-20% of a longitudinal length of the wrapped PTFE extends forwardly of the front end of the mandrel and all values and ranges therebetween), and wherein a longitudinal length of the PTFE tape that is located rearwardly of the front end of the mandrel (i.e., portion of the PTFE tape that is wound about the mandrel) is the desired length of the PTFE tape that is to be positioned about the prosthetic valve prior to the crimping of the prosthetic valve, d) optionally rolling a textured surface against the outer surface of the PTFE tape that is wrapped about the mandrel to increase the mechanical adherence of overlapping portions of the PTFE on the mandrel so as to improve the robustness of the wrapped PTFE tape, e) optionally pulling the portion of PTFE tape that extends forwardly of the front end of the mandrel away from the front end of the mandrel in a direction that is parallel or close to parallel to a longitudinal axis of the mandrel so as to stretch the PTFE tape in a direction that perpendicular or near perpendicular (0-20° to perpendicular and all values and ranges therebetween) to the direction of extrusion of the PTFE tape, thereby resulting in the PTFE tape stretching in such direction, and wherein the front end of the mandrel constricts the stretching of the PTFE tape that is positioned rearward of the front end of the mandrel so as to create a wrapped PTFE tape assembly that has a front portion that has a smaller diameter than the diameter of the rear portion of the PTFE tape assembly that is located on the mandrel, and wherein if the PTFE tape assembly includes a flap or tab then the flap or tab is positioned at the front end portion of the PTFE tape assembly, f) removing the PTFE assembly from the mandrel, and wherein the PTFE tape has a front portion with a smaller diameter than the rear portion to create a “hat shaped” PTFE tape assembly, g) positioning the rear portion of “hat” shaped PTFE tape assembly over a portion of the prosthetic valve, and wherein the smaller diameter or cross-sectional area front portion of the PTFE tape assembly prevents the front portion of the PTFE tape portion from being positioned over or moved over the prosthetic valve, thereby ensuring that the designed length of the rear portion of the PTFE tape assembly that has the larger cross-sectional area or diameter overlies the prosthetic valve.

Another and/or alternative non-limiting object of the present disclosure is the provision of a method for reducing the outer diameter of a crimped prosthetic valve further including the step of removing the polymer tape from the frame after the step of reducing the outer diameter of the prosthetic valve.

Another and/or alternative non-limiting object of the present disclosure is the provision of a method for reducing the outer diameter of a crimped prosthetic valve wherein the polymer tape is PTFE tape; the polymer tape has a width of 0.1-3 inches; the polymer tape has a thickness of 1-10 mils.

Another and/or alternative non-limiting object of the present disclosure is the provision of a method for reducing the outer diameter of a crimped prosthetic valve wherein the PTFE tape is partially or fully non-sintered.

Another and/or alternative non-limiting object of the present disclosure is the provision of a method for reducing the outer diameter of a crimped prosthetic valve wherein the PTFE tape is formed into a sleeve.

Another and/or alternative non-limiting object of the present disclosure is the provision of a method for reducing the outer diameter of a crimped prosthetic valve wherein at least two layers of the polymer tape are wrapped about at least a portion of the frame.

Another and/or alternative non-limiting object of the present disclosure is the provision of a method for reducing the outer diameter of a crimped prosthetic valve wherein at least a portion of the polymer tape is not wrapped about and/or positioned about any portion of the prosthetic valve so as to form a tail of polymer tape; the tail of polymer tape is configured to facilitate in removal of the polymer tape from the frame after the outer diameter of the prosthetic valve has been reduced.

Another and/or alternative non-limiting object of the present disclosure is the provision of a method for reducing the outer diameter of a crimped prosthetic valve wherein 30-100% of a longitudinal length of the frame includes the polymer tape wrapped about the outer surface of the frame.

Another and/or alternative non-limiting object of the present disclosure is the provision of a method for reducing the outer diameter of a crimped prosthetic valve wherein the leaflet protector further includes a compressible foam sleeve; the compressible foam sleeve includes an internal cavity that extends at least 50% of a longitudinal length of the compressible foam sleeve; and further including the step of inserting the compressible foam sleeve about at least a portion of an outer surface of the prosthetic valve such that at least a portion of an outer surface of the polymer tape that is wrapped about the outer surface of the frame is positioned in the internal cavity of the compressible foam sleeve.

Another and/or alternative non-limiting object of the present disclosure is the provision of a method for reducing the outer diameter of a crimped prosthetic valve wherein the compressible foam sleeve is positioned about at least a portion of an outer surface of the prosthetic valve prior to the step of reducing the outer diameter of the prosthetic valve.

Another and/or alternative non-limiting object of the present disclosure is the provision of a method for reducing the outer diameter of a crimped prosthetic valve further including the steps of i) removing the compressible foam sleeve after the outer diameter of the prosthetic valve has been reduce; ii) subjecting the prosthetic valve to further diameter reduction while the polymer tape remains on the frame and after the compressible foam sleeve has been removed from the prosthetic valve; and iii) removing the polymer tape from the frame after a final diameter reduction of the prosthetic valve is completed.

Another and/or alternative non-limiting object of the present disclosure is the provision of a method for reducing the outer diameter of a crimped prosthetic valve wherein the prosthetic valve includes an inner skirt and/or an outer skirt that is positioned along 10-60% of a longitudinal length of the frame (and all values and ranges therebetween); the polymer tape is wrapped about 0-100% of an outer surface (and all values and ranges therebetween) of the inner skirt and/or the outer skirt.

Another and/or alternative non-limiting object of the present disclosure is the provision of a prosthetic valve system configured to enable an outer diameter of a prosthetic valve to be reduced while limiting damage to one or more components of the prosthetic valve; the prosthetic valve system comprising: a) the prosthetic valve; the prosthetic valve includes a frame, a leaflet structure supported by the frame; and b) a leaflet protector that is configured to inhibit or prevent damage to the leaflet structure during crimping the prosthetic valve; the leaflet protector includes a flexible layer of first polymer tape; the layer of the first polymer tape is wrapped at least once about an outer surface of the frame prior to reducing the outer diameter of the prosthetic valve.

Another and/or alternative non-limiting object of the present disclosure is the provision of a prosthetic valve system configured to enable an outer diameter of a prosthetic valve to be reduced while limiting damage to one or more components of the prosthetic valve wherein the PTFE tape is formed into a sleeve that is positioned about all or a portion of the prosthetic valve prior to the crimping of the prosthetic valve.

Another and/or alternative non-limiting object of the present disclosure is the provision of a prosthetic valve system configured to enable an outer diameter of a prosthetic valve to be reduced while limiting damage to one or more components of the prosthetic valve wherein the layer of the first polymer tape is wrapped at least twice about at least a portion of the frame.

Another and/or alternative non-limiting object of the present disclosure is the provision of a prosthetic valve system configured to enable an outer diameter of a prosthetic valve to be reduced while limiting damage to one or more components of the prosthetic valve wherein at least a portion of the first polymer tape is not wrapped about any portion of the prosthetic valve so as to form a tail of first polymer tape; the tail of first polymer tape is configured to facilitate in removal of the first polymer tape from the frame after the outer diameter of the prosthetic valve has been reduced.

Another and/or alternative non-limiting object of the present disclosure is the provision of a prosthetic valve system configured to enable an outer diameter of a prosthetic valve to be reduced while limiting damage to one or more components of the prosthetic valve wherein 30-100% of a longitudinal length of the frame includes the first polymer tape wrapped about the outer surface of the frame.

Another and/or alternative non-limiting object of the present disclosure is the provision of a prosthetic valve system configured to enable an outer diameter of a prosthetic valve to be reduced while limiting damage to one or more components of the prosthetic valve wherein the leaflet protector includes the layer of the first polymer tape and a layer of compressible material; the layer of first polymer tape is connected to a first side of the compressible material; the compressible material is formed of a different material from the first polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein a thickness of the compressible material is greater than a thickness of the layer of the first polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein a compressibility of the compressible material is greater than a compressibility of the layer of the first polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein leaflet protector further includes a layer of a second polymer tape; the layer of the second polymer tape is connected to a second side of the compressible material.

Another and/or alternative non-limiting object of the present disclosure is the provision of a prosthetic valve system configured to enable an outer diameter of a prosthetic valve to be reduced while limiting damage to one or more components of the prosthetic valve wherein a thickness of the compressible material is greater than a thickness of the layer of the second polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein a compressibility of the compressible material is greater than a compressibility of the layer of the second polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein the layer of the second polymer tape is a) the same materials as the layer of the first polymer tape, and/or b) a same thickness as the layer of the first polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein the leaflet protector further includes a compressible foam sleeve; the compressible foam sleeve includes an internal cavity that extends at least 50% of a longitudinal length of the compressible foam sleeve; the compressible foam sleeve is inserted about at least a portion of an outer surface of the prosthetic valve; at least a portion of an outer surface of the first polymer tape is positioned in the internal cavity of the compressible foam sleeve when the compressible foam sleeve is inserted about at least a portion of an outer surface of the prosthetic valve.

Another and/or alternative non-limiting object of the present disclosure is the provision of a prosthetic valve system configured to enable an outer diameter of a prosthetic valve to be reduced while limiting damage to one or more components of the prosthetic valve wherein the leaflet protector is configured such that a) both the compressible foam sleeve and the first polymer tape are simultaneously positioned about at least a portion of an outer surface of the prosthetic valve, or b) the first polymer tape is configured to be positioned about at least a portion of an outer surface of the prosthetic valve prior to inserting the compressible foam sleeve about at least a portion of an outer surface of the prosthetic valve.

Another and/or alternative non-limiting object of the present disclosure is the provision of a prosthetic valve system configured to enable an outer diameter of a prosthetic valve to be reduced while limiting damage to one or more components of the prosthetic valve wherein a thickness of a wall of the compressible foam sleeve is at least 25% thicker than the layer of the first polymer tape.

Another and/or alternative non-limiting object of the present disclosure is the provision of a prosthetic valve system configured to enable an outer diameter of a prosthetic valve to be reduced while limiting damage to one or more components of the prosthetic valve wherein the compressible foam sleeve is at least partially form of a polyurethane foam.

Another and/or alternative non-limiting object of the present disclosure is the provision of a prosthetic valve system configured to enable an outer diameter of a prosthetic valve to be reduced while limiting damage to one or more components of the prosthetic valve wherein 75-100% of a longitudinal length of the prosthetic valve is positioned in the cavity of the compressible foam sleeve.

Another and/or alternative non-limiting object of the present disclosure is the provision of a prosthetic valve system configured to enable an outer diameter of a prosthetic valve to be reduced while limiting damage to one or more components of the prosthetic valve wherein the prosthetic valve includes an inner skirt and/or an outer skirt that is positioned along 10-60% of a longitudinal length of the frame; the first polymer tape is wrapped about 0-30% of an outer surface of the inner skirt and/or the outer skirt.

Another and/or alternative non-limiting object of the present disclosure is the provision of a device and method for crimping of the frame of the prosthetic valve includes the use of a leaflet folding device that can optionally be used with the crimper device and/or the leaflet protector to a) facilitate in obtaining a leaflet folding configuration during the crimping of the frame of a prosthetic valve so as to obtain a desired folded leaflet configuration after the frame has been fully crimped, and b) to inhibit or prevent damage to one or more leaflets during the crimping of the medical device.

Another and/or alternative non-limiting object of the present disclosure is the provision of a device and method for crimping a crimpable or plastically deformable portion of a medical device that includes one or more leaflets that has a desired folding pattern so as to reduce void spaces about the leaflets after completion of the crimping of the crimpable or plastically deformable portion of a medical device.

Another and/or alternative non-limiting object of the present disclosure is the provision of a device and method for crimping a crimpable or plastically deformable portion of a medical device that includes one or more leaflets so as to facilitate in the desired folding profile of the leaflets of a prosthetic valve during the crimping of the frame of the prosthetic valve to reduce the number and volume of void spaces between and about the leaflets and to reduce the crimped outer diameter profile of the prosthetic heat valve, while reducing the risk of damage to the leaflets and other components of the prosthetic valve during the crimping process.

Another and/or alternative non-limiting object of the present disclosure is the provision of a method for reducing the outer diameter of a crimped prosthetic valve comprising: a) providing a prosthetic valve; the prosthetic valve includes a frame, a leaflet structure supported by the frame; the frame in a non-fully crimped orientation; b) providing a diameter reducing device that is configured to reduce an outer diameter of the prosthetic valve; c) providing a leaflet protector that is configured to inhibit or prevent damage to the leaflet structure during crimping the prosthetic valve; the leaflet protector including a flexible polymer tape; d) wrapping at least one layer of the polymer tape about an outer surface of the frame; e) at least partially inserting the prosthetic valve in the diameter reducing device; and f) reducing an outer diameter of the prosthetic valve by use of the diameter reducing device; and wherein the leaflet protector inhibits or prevents the leaflet structure from moving into one or more openings in the frame as the outer diameter of the prosthetic valve is reduced.

Another and/or alternative non-limiting object of the present disclosure is the provision of a prosthetic valve system configured to cause an outer diameter or cross-sectional area of a prosthetic valve to be reduced while limiting damage to one or more components of the prosthetic valve; the prosthetic valve system comprises a prosthetic valve; the prosthetic valve includes a frame, and a leaflet structure supported by the frame; and a leaflet protector that is configured to inhibit or prevent damage to the leaflet structure during crimping the frame of the prosthetic valve; the leaflet protector includes a flexible layer of first polymer tape; the layer of the first polymer tape is wrapped at least once about an outer surface of at least a portion of the frame prior to fully reducing the outer diameter or cross-sectional area of the frame of the prosthetic valve.

Another and/or alternative non-limiting object of the present disclosure is the provision of a prosthetic valve system wherein the first polymer tape is non-sintered PTFE tape.

Another and/or alternative non-limiting object of the present disclosure is the provision of a prosthetic valve system wherein the layer of the first polymer tape is wrapped at least 1.1 times about at least a portion of the frame.

Another and/or alternative non-limiting object of the present disclosure is the provision of a prosthetic valve system wherein at least a portion of the first polymer tape is not wrapped about any portion of the prosthetic valve so as to form a tail on first polymer tape; the tail is configured to facilitate in removal of the first polymer tape from the frame after the outer diameter or cross-sectional area of the frame of the prosthetic valve has been reduced; the tail constitutes at least 2% of a longitudinal length of the first polymer tape.

Another and/or alternative non-limiting object of the present disclosure is the provision of a prosthetic valve system wherein 30-100% of a longitudinal length of the frame includes the first polymer tape wrapped about the outer surface of the frame.

Another and/or alternative non-limiting object of the present disclosure is the provision of a prosthetic valve system wherein the prosthetic valve includes an outer skirt; 0-20% of a longitudinal length of the outer skirt as measured along a longitudinal axis of the frame includes the first polymer tape wrapped about the outer surface of the outer skirt.

Another and/or alternative non-limiting object of the present disclosure is the provision of a prosthetic valve system wherein the leaflet protector includes the layer of the first polymer tape and a layer of compressible material; the layer of first polymer tape is located on a first side of the compressible material when the leaflet protector is positioned about the frame; the compressible material is formed of a different material from the first polymer tape; a thickness of the compressible material is greater than a thickness of the layer of the first polymer tape; a compressibility of the compressible material is greater than a compressibility of the layer of the first polymer tape.

Another and/or alternative non-limiting object of the present disclosure is the provision of a prosthetic valve system wherein the first polymer tape is connected to the first side of the layer of compressible material.

Another and/or alternative non-limiting object of the present disclosure is the provision of a prosthetic valve system wherein the first polymer tape is releasably positioned on and/or releasably connected to the first side of the layer of compressible material.

Another and/or alternative non-limiting object of the present disclosure is the provision of a prosthetic valve system wherein leaflet protector further includes a layer of a second polymer tape; the layer of the second polymer tape is positioned on a second side of the compressible material; the compressible material is formed of a different material from the second polymer tape; a thickness of the compressible material is greater than a thickness of the layer of the second polymer tape; a compressibility of the compressible material is greater than a compressibility of the layer of the second polymer tape; the second polymer tape is a) a same material as the layer of the first polymer tape, and/or b) a same thickness as the layer of the first polymer tape.

Another and/or alternative non-limiting object of the present disclosure is the provision of a prosthetic valve system wherein the second polymer tape is connected to the second side of the layer of compressible material.

Another and/or alternative non-limiting object of the present disclosure is the provision of a prosthetic valve system wherein the second polymer tape is releasably positioned on and/or releasably connected to the second side of the layer of compressible material.

Another and/or alternative non-limiting object of the present disclosure is the provision of a prosthetic valve system wherein the compressible material is a compressible foam sleeve; the compressible foam sleeve includes an internal cavity that extends at least 50% of a longitudinal length of the compressible foam sleeve; and further including the step of inserting the compressible foam sleeve about at least a portion of an outer surface of the prosthetic valve; at least a portion of the first polymer tape is positioned in the internal cavity of the compressible foam sleeve when the compressible foam sleeve is inserted about at least a portion of an outer surface of the prosthetic valve.

Another and/or alternative non-limiting object of the present disclosure is the provision of a prosthetic valve system wherein further including the step of positioning the compressible foam sleeve about at least a portion of the outer surface of the first polymer tape after the first polymer tape has been applied to at least a portion of the outer surface of the frame.

Another and/or alternative non-limiting object of the present disclosure is the provision of a prosthetic valve system wherein further including the steps of i) removing the compressible foam sleeve after the outer diameter or cross-sectional area of the prosthetic valve has been reduce; ii) subjecting the prosthetic valve to further diameter reduction or cross-sectional area reduction while the first polymer tape remains on the frame and after the compressible foam sleeve has been removed from the prosthetic valve; and iii) removing the first polymer tape from the frame after a further diameter reduction or cross-sectional reduction of the frame of the prosthetic valve.

Another and/or alternative non-limiting object of the present disclosure is the provision of a method for forming a preformed leaflet protector comprising: a) providing a strip of flexible polymer material; b) providing a mandrel, c) wrapping the strip of polymer material at least 1.25 times about an outer perimeter of the mandrel; d) removing the wrapped polymer strip from the mandrel to form the leaflet protector; and e) wherein the leaflet protector includes a body that has a cavity; the cavity extends 50-100% of a longitudinal length of the body; the cavity is formed by overlapping portions of the strip of polymer material.

Another and/or alternative non-limiting object of the present disclosure is the provision of a method for forming a preformed leaflet protector wherein the strip of polymer material is one or more of a) a PTFE tape, b) a width of 0.1-3 inches, c) a thickness of 1-10 mils, and/or d) is non-sintered PTFE tape.

Another and/or alternative non-limiting object of the present disclosure is the provision of a method for forming a preformed leaflet protector wherein the step of wrapping includes wrapping only a portion of the strip about the mandrel such that a proximal portion of the wrapped portion of the strip extends forwardly of a front end of the mandrel; and further including the step of pulling the proximal portion of the wrapped portion of the strip that extends forwardly of a front end of the mandrel away from the mandrel along a longitudinal axis of the mandrel to cause a reduction in diameter or cross-sectional area of the cavity in the proximal portion.

Another and/or alternative non-limiting object of the present disclosure is the provision of a method for forming a preformed leaflet protector wherein an average diameter or cross-sectional area of the cavity in the proximal portion of the preformed leaflet protector is less than an average diameter or cross-sectional area of a proximal portion of the cavity that is formed by the wrapped portion of the strip that is located about the mandrel.

Another and/or alternative non-limiting object of the present disclosure is the provision of a method for forming a preformed leaflet protector wherein a longitudinal length of the cavity in the proximal portion of the of the preformed leaflet protector is longer than a longitudinal length of the cavity in the distal portion of the preformed leaflet protector.

Another and/or alternative non-limiting object of the present disclosure is the provision of a method for forming a preformed leaflet protector wherein the longitudinal length of the cavity in the proximal portion of the preformed leaflet protector is 5-300% longer than the longitudinal length of the cavity in the distal portion of the preformed leaflet protector.

Another and/or alternative non-limiting object of the present disclosure is the provision of a method for forming a preformed leaflet protector wherein the average diameter or cross-sectional area of cavity in the proximal portion of the preformed leaflet protector is 5-300% larger than the average diameter or cross-sectional area of cavity in the distal portion of the preformed leaflet protector.

Another and/or alternative non-limiting object of the present disclosure is the provision of a method for forming a preformed leaflet protector further including the step of applying compression to one or more regions of the strip of polymer material that is wrapped about the mandrel to form a plurality of compression regions on the strip of polymer material; the plurality of compression regions configured to facilitate in bonding together the overlapping portions of the strip of polymer material that is located about the mandrel.

Another and/or alternative non-limiting object of the present disclosure is the provision of a method for forming a preformed leaflet protector further including the step of cutting the strip of polymer material prior to the step of wrapping to form the strip of polymer material that has a larger width portion and a smaller width portion; the larger width portion is configured to form a flap or tail on the preformed leaflet protector; at least 60% of a longitudinal length of the smaller width portion is configured to form the body; a longitudinal length of the smaller width portion is longer than a longitudinal length of the larger width portion.

Another and/or alternative non-limiting object of the present disclosure is the provision of a method for crimping a valve comprising: a) providing a prosthetic valve; the prosthetic valve includes a frame and a leaflet; the frame has a frame cavity; the frame has a proximal portion that has a proximal end and a distal portion that has a distal end; the frame is configured to be crimped from an expanded orientation to a crimped orientation; a diameter or cross-sectional area of the frame in the expanded orientation is greater than a diameter or cross-sectional area of the frame in the crimped orientation; the frame includes a plurality of frame cells; the leaflet is connected to the frame; 50-100% of the leaflet is positioned in the frame cavity; b) providing a preformed; said preformed leaflet protector includes a body that has a cavity; said preformed leaflet protector has a proximal and distal portion; said cavity extends 50-100% of a longitudinal length of said body; said cavity is formed by overlapping portions of a polymer tape; an average diameter or cross-sectional area of said cavity in said distal portion of said preformed leaflet protector is less than an average diameter or cross-sectional area of said cavity in said proximal portion of said preformed leaflet protector; c) inserting said proximal portion of said preformed leaflet protector about an outer surface of said distal portion of said frame; said average diameter or cross-sectional area of said cavity in said proximal portion of said preformed leaflet protector is equal to or greater than an outer diameter or outer cross-sectional area of said frame in said expanded orientation; said average diameter or cross-sectional area of said cavity in said distal portion of said preformed leaflet protector is less than said outer diameter or outer cross-sectional area of said frame in said expanded orientation such that said cavity in said distal portion of said preformed leaflet protector cannot pass over said distal end of said frame; d) providing a crimper device that is configured to crimp said frame; e) inserting at least a portion of said prosthetic valve that includes said preformed leaflet protector that is positioned about at least a portion of said frame into said crimper device; f) using said crimper device to crimp said frame from said expanded orientation to said crimped orientation; e) removing said prosthetic valve from said crimper device; h) removing at least a portion of said preformed leaflet protector from said frame while said frame is in said crimped orientation; and wherein said preformed leaflet protector inhibits or prevent at least a portion of said leaflet from passing into and/or through one or more of said cells of said frame as said frame is crimped from said expanded orientation to said crimped orientation damage, thereby inhibiting or preventing damage to said leaflet.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1A is a picture of a non-limiting TAV in an expanded orientation in accordance with the present disclosure.

FIG. 1B is another picture of the TAV of FIG. 1A.

FIG. 2A is picture of a portion of a prior art catheter that includes a prior art TAV that is crimped on a portion of the catheter.

FIGS. 2B-2D illustrate a common TAVR procedure for inserting the TAV into a valve of a heart.

FIGS. 3-4 illustrate a non-limiting crimper device that can be used to crimp a TAV.

FIGS. 5 and 6 are pictures of non-limiting folding profiles of leaflets in a partially or fully crimped TAV.

FIG. 7 is a picture of a crimped TAV wherein portions of the leaflet have been compressed between the openings in the frame of the TAV.

FIG. 8 is a picture of a user holding a TAV prior to being crimped, and wherein a leaflet protector that includes one or more layers of a temporary/removeable barrier material in the form of PTFE tape is wrapped about a portion of the frame of the TAV, and wherein an optional tail or flap is formed on one end of the PTFE tape to facilitate in easier removal of the PTFE tape from the TAV after the TAV has been crimped.

FIG. 9 is a picture of a TAV prior to be crimped that includes a leaflet protector that includes of one or more layers of a temporary/removeable barrier material in the form of PTFE tape that is wrapped about a portion of the frame of the TAV, and wherein an optional tail or flap is formed on one end of the PTFE tape to facilitate in easier removal of the PTFE tape from the TAV after the TAV has been crimped, and wherein a compression sleeve in the form of a foam sleeve is positioned about at least a portion of the PTFE tape and at least a portion of the frame of the TAV.

FIG. 10 is picture of a crimped TAV wherein the temporary/removeable barrier material of the leaflet protector has been pressed in between the openings in the frame during the crimping process, thereby inhibiting or preventing portions of the leaflets from being compressed between the openings in the frame of the TAV, and wherein an optional tail or flap is formed on one end of the PTFE tape to facilitate in easier removal of the PTFE tape from the TAV after the TAV has been crimped.

FIG. 11 is a picture of a TAV that after being crimped on an inflatable balloon of a balloon catheter on a delivery device, and wherein the temporary/removeable barrier material of the leaflet protector remains on the TAV during the crimping process of the TAV on the inflatable balloon, thereby inhibiting or preventing portions of the leaflets from being compressed between the openings in the frame of the TAV, and wherein an optional tail or flap is formed on one end of the PTFE tape to facilitate in easier removal of the PTFE tape from the TAV after the TAV has been crimped.

FIG. 12 is a picture of a TAV after being crimped on an inflatable balloon on a delivery catheter, and after the temporary/removeable barrier material of the leaflet protector has been removed from the TAV, and illustrates that no portion of the leaflets has been trapped, compressed or damaged between the openings in the frame of the TAV.

FIGS. 13 and 14 are pictures of another non-limiting leaflet protector that includes a compression sleeve in the form of a foam sleeve that has a cavity therethrough, and one or more layers of a temporary/removeable barrier material in the form of PTFE tape that are positioned in the cavity of the compression sleeve, and wherein the temporary/removeable barrier material optionally includes one or more tabs extending out from the cavity of the compression sleeve, and wherein the compression sleeve and the temporary/removeable barrier material are configured to be positioned about the TAV while in the expanded orientation and prior to the frame being crimped.

FIGS. 15 and 16 illustrate another non-limiting leaflet protector that includes a layer of temporary/removeable barrier material in the form of PTFE tape that is positioned on one or both sides of a layer of compressible layer in the form of a foam material, and wherein the layer of compressible material and the one or more layers of temporary/removeable barrier material are configured to be positioned about the TAV while in the expanded orientation and prior to the frame being crimped.

FIG. 17 is a picture of a piece of PTFE tape that has been cut to form a tail or flap at one end of the PTFE tape.

FIG. 18 is a picture of the cut piece of PTFE tape of FIG. 17 that has been wrapped about a cylindrical mandrel, and wherein the tail or flap of the PTFE tape is partially or fully positioned forwardly of the front end of the mandrel.

FIG. 19 is a picture of a portion of the piece of PTFE tape that is positioned about the mandrel of FIG. 18 that has been optionally textured to improve adherence of the overlapping portions of the PTFE tape that are positioned about the mandrel.

FIG. 20 is a picture of the portion of the PTFE tape that is positioned forwardly of the front end of the mandrel that has been pulled forwardly along the longitudinal axis of the mandrel to cause stretching of a portion of the PTFE tape and the formation of a smaller diameter or cross-sectional area of the cavity of the PTFE tape that is located forwardly of the front end of the mandrel as compared to the portion of the PTFE tape that is wrapped about the mandrel.

FIGS. 21 and 22 are pictures of two different views of the formed PTFE tape of FIG. 20 after being removed from the mandrel, and after the formed PTFE tape has been position about a portion of the TAV prior to the partial or full crimping of the TAV.

### DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values), and also include all ranges therebetween.

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

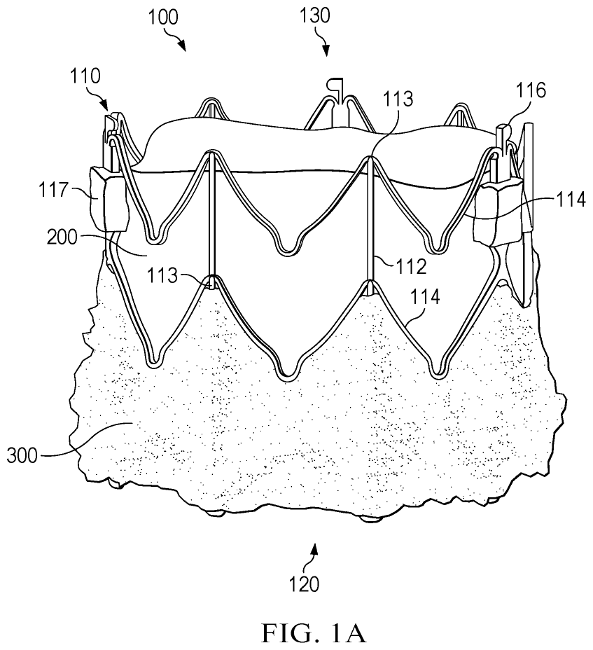

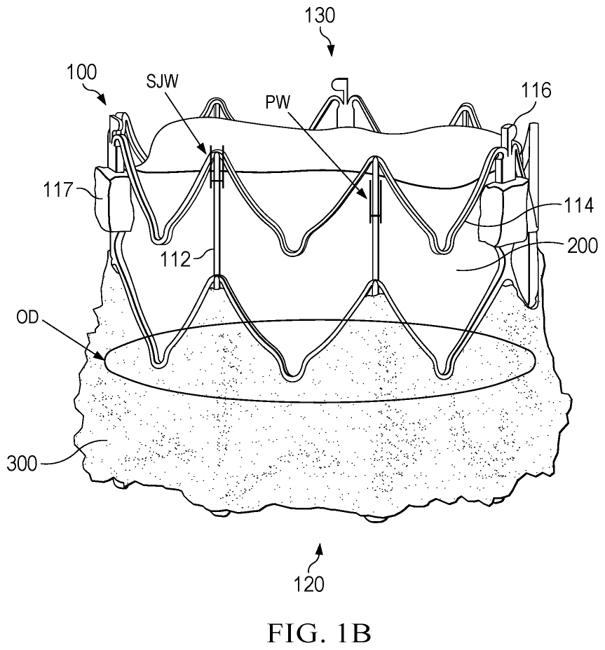

Referring now to FIGS. 1A and 1B, these figures are illustrations of a non-limiting implantable prosthetic valve 100 (e.g., TAV) in accordance with the present disclosure. FIGS. 2A-2C illustrate a prosthetic valve 100 that has been crimped about a distal region of a catheter C and a non-limiting method for inserting the prosthetic valve 100 in a valve region A (e.g., aortic valve, etc.) of a heart H. Prosthetic valve 100 can be implanted in the annulus of native aortic valve A; however, prosthetic valve 100 also can be configured to be implanted in other valves of the heart or other regions of the heart. Although the medical device illustrated is a TAV, the present disclosure is not limited to TAVs or any other heart valve replacement devices.

Referring again to FIGS. 1A-1B, prosthetic valve 100 generally comprises a frame 110 formed of a plurality of vertically extending axial longitudinal members and angular articulating members 112, 114, strut joints 113, leaflet arrangement 200 supported by frame 110, and an outer skirt 300 secured to the outer surface of frame 110, leaflet arrangement 200, and/or inner skirt (not shown). The prosthetic valve 100 typically includes an inner skirt that is connected to the inner surface of the frame 110, and is also typically connected to leaflet arrangement 200 and the outer skirt 300. The frame can optionally include one or more orientation structures or commissural markers 116. The one or more commissural markers 116 are generally is located on the top end of one or more of the vertically extending axial longitudinal members 112. As illustrated in FIGS. 1A and 1B, the strut joints 113 do not extend above the top ends of the vertically extending axial longitudinal members 112 or below the bottom ends of the vertically extending axial longitudinal members 112; however, this is not required. Likewise, the angular articulating members 112, 114 do not extend above the top ends of the vertically extending axial longitudinal members 112 or below the bottom ends of the vertically extending axial longitudinal members 112; however, this is not required. As also illustrated in FIGS. 1A and 1B, each of the commissural markers 116 extends above the top end of the vertically extending axial longitudinal members 112.

Prosthetic valve 100 has a “lower” or proximal end 120 and an “upper” or distal end 130, wherein lower or proximal end 120 of prosthetic valve 100 is the inflow end and the upper or distal end 130 of prosthetic valve 100 is the outflow end.

Frame 110 can be optionally be coated with a polymer material (e.g., silicone, PTFE, ePTFE, polyurethane, polyolefins, hydrogels, biological materials [e.g., pericardium or biological polymers such as collagen, gelatin, or hyaluronic acid derivatives], etc.). The coating can be used to partially or fully encapsulate one or more of the vertically extending axial longitudinal members 112 and/or non-vertically angular articulating members 114 on frame 110 and/or to partially or fully fill-in one or more of the openings between the angular articulating members 114 and/or vertically extending axial longitudinal members 112.

Frame 110 can be optionally coated with a biological agent. As can be appreciated, other or additional components of the prosthetic valve 100 (e.g., outer skirt, inner skirt, leaflets, etc.) can optionally include the biological agent.

Frame 110 can optionally include a nitride or nirided coating.

Frame 110 can be optionally coated with an enhancement coating (e.g., chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}) [e.g., cubic ZrN:O, cubic ZrO₂:N, tetragonal ZrO₂:N, and monoclinic ZrO₂: N phase coatings], oxyzirconium-nitrogen-carbon (ZrNC), zirconium OxyCarbide (ZrOC), etc.). As can be appreciated, other or additional components of the prosthetic valve 100 (e.g., outer skirt, inner skirt, leaflets, etc.) can optionally include the enhancement coating. The enhancement coating can optionally include a metal adhesion layer (e.g., titanium layer, zirconium layer, etc.). As defined herein, the enhancement coating, when used, is not a) a coating of an agent or biological agent, b) a nitride or nirided coating, c) a coating of polymer material, or d) a solely plated or coated metal layer.

The outer skirt 300 can be formed of a variety of flexible materials (e.g., polymer (e.g., polyethylene terephthalate (PET), polyester, nylon, Kevlar, silicon, etc.), composite material, metal, fabric material, etc.

Outer skirt 300 can be used to 1) at least partially seal and/or prevent perivalvular leakage, 2) at least partially protect one or more of the leaflets of leaflet arrangement 200 from damage during the crimping process of prosthetic valve 100, and/or 3) at least partially protect one or more of the leaflets of leaflet arrangement 200 from damage during the operation of prosthetic valve 100 in heart H. In one non-limiting configuration, the outer skirt 300 is only connected to the inner skirt and frame 110, and wherein the outer skirt 300 is optionally only connected to the vertically extending axial longitudinal members 112 of the frame 110, but not the angular articulating members 114 of the frame 110.

The inner skirt generally is positioned completely around a portion of the inner surface of frame 110. Generally, the inner skirt is positioned about the inner surface of the lower portion of frame 110 and does not fully cover the upper portion of the inner surface of frame 110; however, this is not required. The inner skirt can be connected to frame 110, the outer skirt 300, and/or the leaflet arrangement 200 by a variety of arrangements (e.g., sutures, adhesive, melted connection, stitches, clamping arrangement, etc.). In one non-limiting configuration, the inner skirt is connected to frame 110, the outer skirt 300, and the leaflets of the leaflet arrangement 200, and wherein the inner skirt is optionally only connected to the lower portion of the leaflets of the leaflet arrangement 200, and wherein the inner skirt is optionally connected to both the vertically extending axial longitudinal members 112 of the frame 110 and the angular articulating members 114 of the frame 110, and wherein the inner skirt is optionally connected to only on row of angular articulating members 114 of the frame 110. In another non-limiting configuration, the bottom portion of the inner skirt is optionally connected to the bottom portion of the outer skirt 300, and wherein the bottom portion of the inner skirt and the bottom portion of the outer skirt 300 are optionally not connected to the frame 110, and the leaflets of the leaflet arrangement 200 are connected to the inner skirt at a location that is optionally above the connection location of the bottom portion of the inner skirt and the bottom portion of the outer skirt 300, and wherein a top portion of the inner skirt is connected to one row of angular articulating members 114 of the frame 110, and wherein the row of angular articulating members 114 of the frame 110 is that the inner skirt is connected thereto is optionally not the top row of angular articulating members 114 of the frame 110, and wherein a top portion of the outer skirt 300 is optionally connected to the inner skirt at a location that is below the angular articulating members 114 of the frame 110 that the inner skirt is connected thereto, and wherein the top profile of the inner skirt optionally has the same or similar shape as the profile of the angular articulating members 114 of the frame 110 to which the inner skirt is connected thereto, and wherein the top profile of the outer skirt 300 has the same or similar profile to the top profile of the inner skirt to which the outer skirt 300 is connected thereto. At least a portion of the inner skirt can optionally be located on the exterior surface of frame 110; however, this is not required.

Leaflet arrangement 200 can be attached to frame 110 and/or the inner skirt. The connection arrangement used to secure leaflet arrangement 200 to frame 110 and/or the inner skirt is non-limiting (e.g., sutures, melted bold, adhesive, stitching, clamp arrangement, etc.). The material used to form the one or more leaflets of leaflet arrangement 200 include, but are not limited to, polymers, bovine pericardial tissue, bovine tissue, porcine tissue, biocompatible synthetic materials, or various other suitable natural or synthetic materials.

Leaflet arrangement 200 can be comprised of two or more leaflets (e.g., 2, 3, 4, 5, 6, etc.). In one non-limiting arrangement, leaflet arrangement 200 includes three leaflets that are arranged to collapse in a tricuspid arrangement. The size, shape and configuration of the one or more leaflets of leaflet arrangement 200 are non-limiting. In one non-limiting arrangement, the leaflets generally have the same shape, size, configuration and thickness.

Two of more of the leaflets of leaflet arrangement 200 can optionally be secured to one another at their adjacent sides to form commissures 220 of leaflet arrangement 200 (the edges where the leaflets come together). Leaflet arrangement 200 can be secured to frame 110 and/or inner skirt by a variety of connection arrangement (e.g., sutures, adhesive, melted bond, clamping arrangement, etc.). In one non-limiting configuration, the one or more leaflets of the leaflet arrangement 200 is connected to frame opening arrangement or commissural opening in the frame 110. In one non-limiting configuration, the commissures 220 of leaflet arrangement 200 is positioned at least partially through the frame opening arrangement or commissural opening and then secured to the frame 110 by one or more connection arrangements (e.g., sutures, adhesive, melted bond, clamping arrangement, etc.).

The frame 110 can includes two or more rows of cells. As illustrated in FIGS. 1A and 1B, frame 110 includes three rows of angular articulating members and two rows of cells wherein each row of cells included nine cells. Each of the cells are open cells; however, this is not required. An open cell is a cell wherein at least one wall of the cell is formed by an angular articulating member and at least one wall of the cell is formed by a vertically extending axial longitudinal member.

Prosthetic valve 100 is configured to be radially collapsible to a collapsed or crimped orientation for introduction into the body on a delivery catheter (FIG. 2A) and radially expandable to an expanded orientation for implanting prosthetic valve 100 at a desired location in heart H (e.g., aortic valve A, etc.) (FIG. 2D). The frame of prosthetic valve 100 is made of a plastically-expandable material that permits crimping of the frame to a smaller profile for delivery and expansion of prosthetic valve 100 using an expansion device. FIG. 2A illustrates a generic frame F of a prosthetic valve that is crimped on a generic balloon catheter C. The inflatable balloon B on the balloon catheter C can be used to expand the frame F from a crimped orientation to an expanded orientation. Various type of crimping apparatus and techniques can be used to crimp the prosthetic valve on the balloon delivery catheter. The process of crimping a prosthetic valve using a crimping device is known in the art and will not be described herein.

As illustrated in FIGS. 2B-2D, once prosthetic valve 100 is crimped on balloon B of a balloon catheter C, balloon catheter C is inserted through a blood vessel and to the location in heart H wherein prosthetic valve 100 is to be deployed (See FIG. 2B). At the treatment location, the inflatable balloon B on balloon catheter C is expanded to thereby cause prosthetic valve 100 to be expanded and secured in a valve region A of heart H (See FIG. 2C). Thereafter, inflatable balloon B is deflated and balloon catheter C is removed from the patient (See FIG. 2D).

The metal alloy frame 110 of the prosthetic valve 100 can optionally be formed of a refractory metal alloy or an alloy that includes at least 5 awt. % rhenium and/or hafnium (e.g., MoRe alloy, ReW alloy, MoReCr alloy, MoReTa alloy, MoReTi alloy, HFMo alloy, HfCr alloy, WCu alloy, ReCr alloy, Mo alloy, Re alloy, W alloy, Ta alloy, Nb alloy); however, other metal alloys can be used (e.g., stainless steel (e.g., 316, 316L), nitinol, TiAlV alloy, CoCr alloy, aluminum alloy, nickel alloy, titanium alloy, tungsten alloy, molybdenum alloy, copper alloy, beryllium-copper alloy, titanium-nickel alloy, rhenium alloy, etc.).

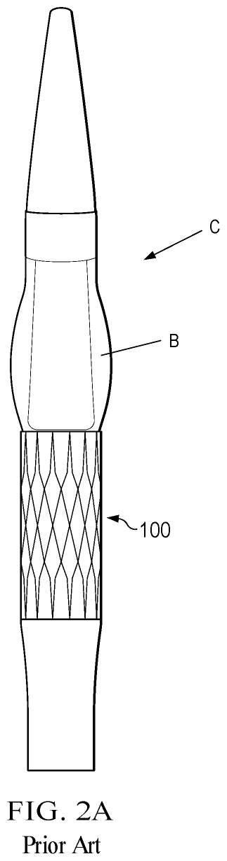

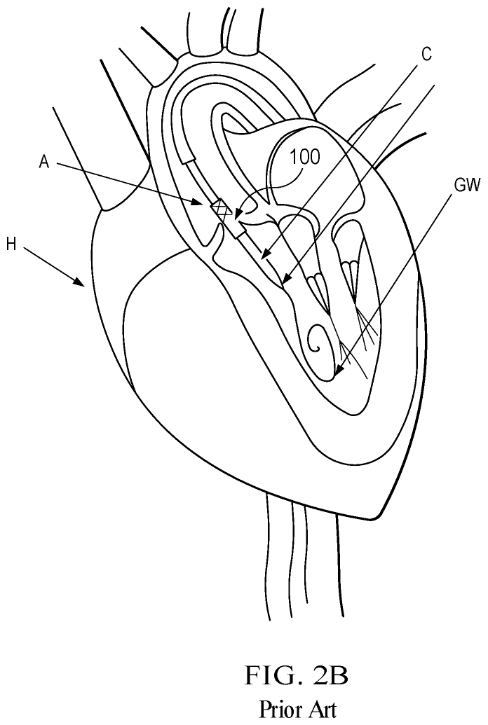

Referring now to FIGS. 3-4, a non-limiting crimper device 50 is illustrated that can be used to crimp the prosthetic valve 100. Crimper device 50 includes an opening 30 wherein the prosthetic valve 100 is placed to be crimped.

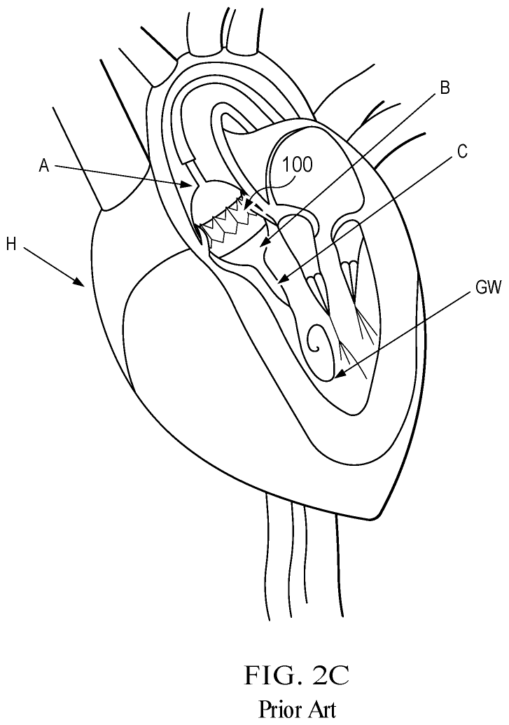

Referring now to FIG. 5, there is illustrated a rear-end view or outflow side end view of a prosthetic valve that is has been partially crimped by a crimper device.

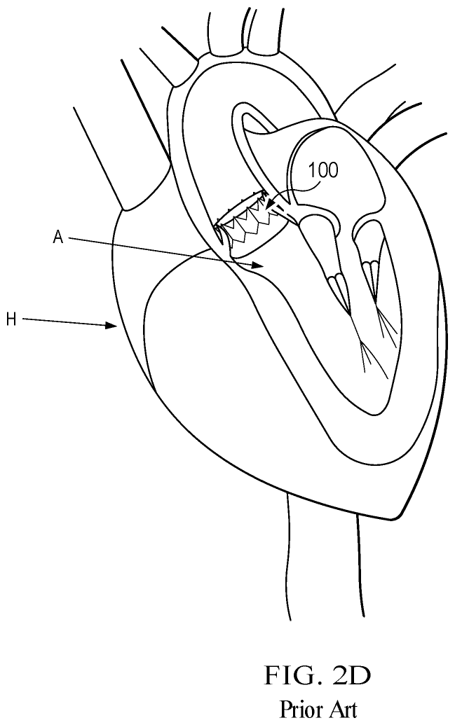

Referring now to FIG. 6, there is illustrated another rear-end view or outflow side end view of another a prosthetic valve showing the folding profile of leaflets in the fully crimped prosthetic valve that has been crimped by the crimper device.

When crimping the prosthetic valve 100 onto the delivery system, the diameter of the frame 110 is reduced from an expanded diameter to a much smaller diameter. As the crimping process progresses, the leaflets of leaflet structure 200 are compressed. As this compression occurs, one or more portions of the leaflets of leaflet structure 200 can be pushed or forced from the inside of the frame 110 through one or more of the frame openings. When one or more portions of the leaflets of leaflet structure 200 are caused to protrude into and/or through one or more openings in the frame 110 as the diameter of the frame 110 is continued to be reduced during the crimping process, the one or more portions of the leaflets of leaflet structure 200 that are positioned in the openings in the frame 110 can be damaged by the pressing of the frame struts or axial longitudinal members 112 and/or non-vertically angular articulating members 114 on frame 110 against the protruding portions of the leaflets. When the one or more portions of the leaflets of leaflet structure 200 are positioned in the openings in the frame 110 and the frame 110 is continued to be crimped, such portions of the leaflets of leaflet structure 200 can be cut, sheared, or crimped or otherwise damaged by the frame 110 during the crimping process.

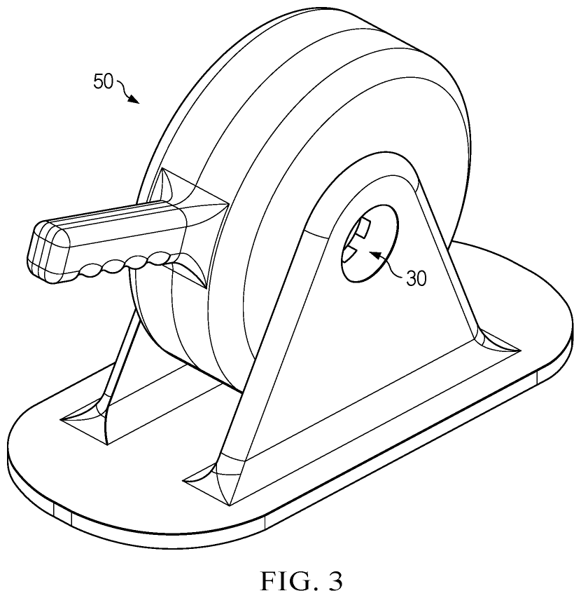

FIG. 7 is an illustration of a partially or fully crimped frame 110 and wherein one or more portions of the leaflets of leaflet structure 200 have been crushed within the openings in the frame 110 and potentially irreparably damaged during the crimping of the frame of the prosthetic valve.

A prior art arrangement to help minimize one or more portions of the leaflets of leaflet structure 200 from being entrapped in the openings in the frame 110 is the use of a compressible foam sleeve that is positioned about the frame 110 prior to the beginning of the crimping process. The foam sleeve is placed around the outer surface of the prosthetic valve 100. During the crimping process, the foam of the foam sleeve is compressed or otherwise forced into the openings of the frame 110, thereby inhibiting or preventing one or more portions of the leaflets of leaflet structure 200 from moving into the openings of the frame 110 during a portion of the crimping process.

One problem associated with the use of the prior art foam sleeve during the crimping process is that large volumes of the foam of the foam sleeve moves into the openings of the frame during the crimping process, which thereby limits the amount the frame can be crimped. In order to address this problem, the foam sleeve is removed from the prosthetic valve 100 prior to being fully crimped, and thereafter, the prosthetic valve 100 is again placed in the crimping device to complete the crimping of the frame of the prosthetic valve 100. After the foam sleeve is removed from the prosthetic valve 100, the frame openings in the frame 110 are typically small and therefore large portions of leaflets of leaflet structure 200 typically do not enter into the frame openings during the further crimping of the prosthetic valve 100. However, smaller amounts of the leaflets of leaflet structure 200 can still be caused to move into and/or through the smaller openings in the frame 110 during the further crimping of the prosthetic valve 100, and thus damage (e.g., pinch, crimp, tear, shear, etc.) such portions of the leaflet structure 200 that have moved into and/or through the smaller openings in the frame 110 during the further crimping of the prosthetic valve 100. Generally, such damage to the portions of the leaflet structure 200 that have moved into and/or through the smaller openings in the frame 110 after the foam sleeve is removed from the prosthetic valve 100 occurs at the edges of the frame 110 and the outer edges of the leaflets of leaflet structure 200; however, it will be appreciated that other regions of the leaflets of leaflet structure 200, and/or damage to the leaflets of leaflet structure 200 can occur in other or additional locations on the frame 110. When one or more of the leaflets of leaflet structure 200 are damaged during the crimp process, such damage can lead to acute (e.g., tears in the leaflet leading to regurgitation, etc.) and/or long-term issues (e.g., Hypoattenuating Leaflet Thickening [HALT]) after the implantation of the prosthetic valve 100 at the treatment site, both of which can adversely impact clinical outcomes for the patient.

In accordance with the present disclosure, the leaflet protector LP is a temporary/removeable barrier material 500. In one non-limiting embodiment, the temporary/removeable barrier material 500 is in the form of or includes polymer tape (e.g., partially or fully non-sintered PTFE tape, etc.) is used to inhibit or prevent one or more portions of the leaflet structure 200 from moving into and/or through the smaller openings in the frame 110 during crimping of the prosthetic valve 100. The temporary/removeable barrier material 500 can be used by itself, or be used in conjunction with a foam sleeve 400. In another non-limiting embodiment, the temporary/removeable barrier material 500 will be described as being in the form of a tape or strip that is wrapped about a portion or all of an outer surface of the prosthetic valve prior to the prosthetic valve being crimped. In another non-limiting embodiment, the temporary/removeable barrier material 500 will be described as being in the form of a preformed sleeve that is positioned about a portion or all of an outer surface of the prosthetic valve prior to the prosthetic valve being crimped. In another non-limiting embodiment, the temporary/removeable barrier material 500 will be described as being used with a foam sleeve 400 during the crimping of the prosthetic valve 100.

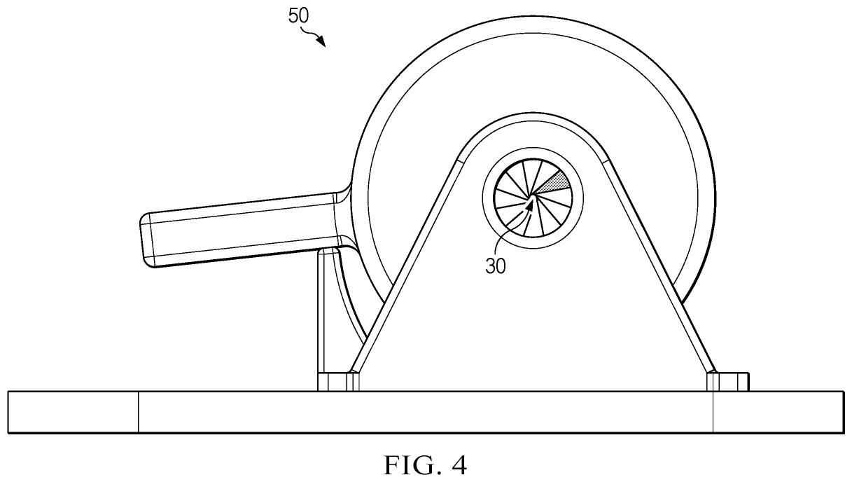

Referring now to FIG. 8, the temporary/removeable barrier material 500 is positioned at least partially about the outer surface of the prosthetic valve 100 prior to the full or partial crimping of the prosthetic valve 100. The temporary/removeable barrier material 500 is selected to be a flexible material that can bend, stretch or otherwise move into the openings in the frame 110 during the crimping process of the prosthetic valve 100 so as to inhibit or prevent damage to the leaflets of leaflet structure 200 by inhibiting or preventing one or more portions of one or more of the leaflets of leaflet structure 200 from moving into or through the openings in the frame 110 during the crimping process of the prosthetic valve 100. The temporary/removeable barrier material 500 is also selected to be a thin material so as to not adversely affect the crimping of the frame 110 during the crimping process of the prosthetic valve 100, thus not interfere with obtaining a small crimped profile of the crimped prosthetic valve. In general, the temporary/removeable barrier material 500 is selected to be a) a flexible material that can be forced into the frame openings during the crimping process of the prosthetic valve 100, b) sufficiently thin to be forced into the frame openings during the crimping process of the prosthetic valve 100, c) robust enough to inhibit or prevent one or more portions of one or more of the leaflets of leaflet structure 200 from moving into the openings in the frame 110 during the crimping process of the prosthetic valve 100, d) strong enough to resist tearing, shearing or otherwise being damaged during the crimp process of the prosthetic valve 100, and/or e) nearly completely removable (e.g., 95-99.999% removable and all values and ranges therebetween) or completely removably from the frame 110 after completion of the crimping process of the prosthetic valve 100.

In one non-limiting configuration, the temporary/removeable barrier material 500 is a flexible polymer tape such as, but not limited to, a non-sintered PTFE tape. The width of the layer of temporary/removeable barrier material 500 is generally at least 0.1 inches (e.g., 0.1-2 inches and all values and ranges therebetween). The thickness of each layer of temporary/removeable barrier material 500 is generally at least 1 mil (e.g., 1-20 mils and all values and ranges therebetween). In one non-limiting configuration, the width of the temporary/removeable barrier material 500, is constant along 50-100% of the longitudinal length of the temporary/removeable barrier material 500. In another non-limiting configuration, the thickness of the temporary/removeable barrier material 500 is constant along 50-100% of the longitudinal length of the temporary/removeable barrier material 500. One or more layers (e.g., 1-10 layers and all values and ranges therebetween) of the temporary/removeable barrier material 500 can be wrapped about a portion or all of the outer surface of the prosthetic valve 100. As illustrated in FIG. 8, the temporary/removeable barrier material 500 is wrapped about the upper portion of the frame 110, and only about a top portion of the outer skirt 300 (e.g., 1-80% and all values and ranges of the longitudinal length or width of the outer skirt). In one non-limiting configuration, the temporary/removeable barrier material 500 is wrapped about 70-100% of the longitudinal length (and all values and ranges therebetween) of the portion of the frame 110 that extends upwardly from the top end of outer skirt 300 and/or inner skirt. In another non-limiting configuration, the temporary/removeable barrier material 500 is wrapped about 1-100% of the longitudinal length (and all values and ranges therebetween) of the portion of the frame 110 that is covered by outer skirt 300 and/or inner skirt. As illustrated in FIG. 8, the temporary/removeable barrier material 500 is wrapped about 1-20% (and all values and ranges therebetween) of the longitudinal length of the portion of the frame 110 that is covered by outer skirt 300. In another non-limiting configuration, the temporary/removeable barrier material 500 is wrapped about 70-100% (and all values and ranges therebetween) of the cross-sectional area of the openings in the frame 110 that are not covered by the outer skirt 300. As illustrated in FIG. 8, the temporary/removeable barrier material 500 is wrapped about 95-100% of the cross-sectional area of the openings in the frame 110 that are not covered by outer skirt 300. As illustrated in FIG. 8, the temporary/removeable barrier material 500 is wrapped 1.1-4 times about the outer surface of the frame 110.

An optional tail 510, handle, flap, tab, or grasping region can be left unwrapped about the prosthetic valve 100. The optional tail 510, handle, flap, tab, or grasping region can be used to facilitate in the removal of the temporary/removeable barrier material 500 from the prosthetic valve 100 after completion of the portion of the crimping process that uses the temporary/removeable barrier material 500. The length of the optional tail 510, handle, flap, tab, or grasping region is non-limiting (e.g., at least 0.1 inches, 0.1-3 inches and all values and ranges therebetween).

After the temporary/removeable barrier material 500 is placed about the prosthetic valve 100, the prosthetic valve 100 and the temporary/removeable barrier material 500 are placed in a crimper 50 to crimp the prosthetic valve 100. During the crimping of the prosthetic valve 100, portions of the temporary/removeable barrier material 500 move into and/or through one or more openings in the frame 110 as the frame 110 is being crimped. The prosthetic valve 100 can be positioned on the catheter C during a portion or all of the crimping process. In one non-limiting process, after the initial crimping of the prosthetic valve 100, the prosthetic valve 100 is removed from the crimper 50 and the crimped prosthetic valve 100 is slid or otherwise position onto or over the inflatable balloon B for final crimping of the prosthetic valve 100.

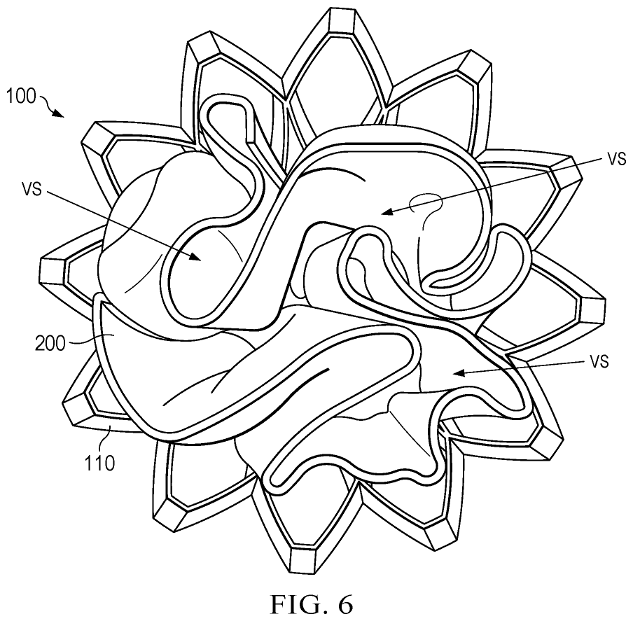

Referring now to FIG. 10, the crimped prosthetic valve 100 is illustrated with the temporary/removeable barrier material 500 remaining about the frame 110, and the temporary/removeable barrier material is shown to be pushed from the outside of the frame 110 partially or fully through the openings in the frame 110. The crimped prosthetic valve 100 is also illustrated as being positioned rearwardly of the inflatable balloon B for optional further crimping prior to being moved over the inflatable balloon B for optional final crimping of the prosthetic valve 100. As can be appreciated, the prosthetic valve can be partially or fully crimped on the inflatable balloon. The movement of the temporary/removeable barrier material 500 partially into or fully through the openings in the frame 110 results in the one or more of the leaflets of leaflet structure 200 being pushed away or otherwise prevented from entering in the openings in the frame 110 during the crimping of the prosthetic valve 100. As illustrated in FIG. 10, the temporary/removeable barrier material 500 was wrapped about the frame 110 such that little, if any, of the temporary/removeable barrier material 500 covers the outer skirt 300; however, this is not required.

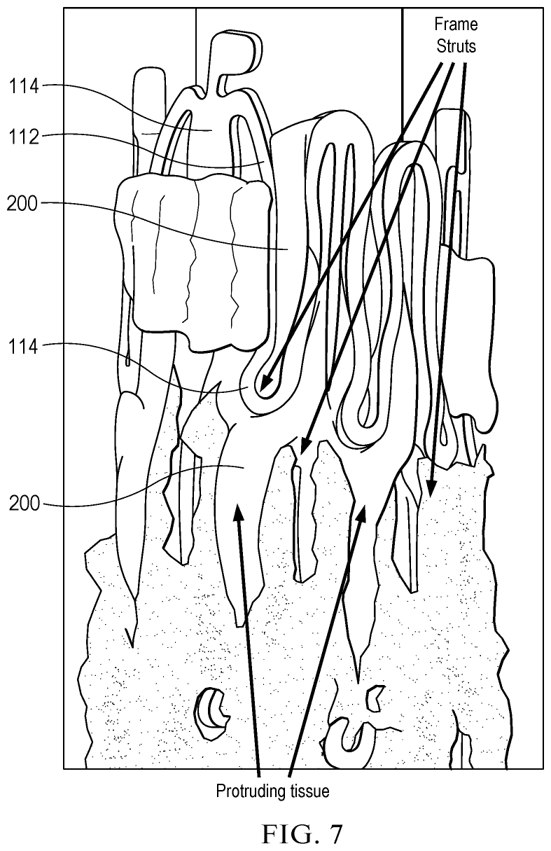

FIG. 11 illustrates the prosthetic valve 100 after it has been fully crimped and crimped about the inflatable balloon B. In the final crimped form, the frame 110 is reduced to its smallest outside diameter and the frame struts or axial longitudinal members 112 and/or non-vertically angular articulating members 114 on frame 110 are positioned closely together. As illustrated in FIGS. 10 and 11, little or none of the one or more of the leaflets of leaflet structure 200 are positioned in the openings in the frame 110 after the crimping of the prosthetic valve 100.

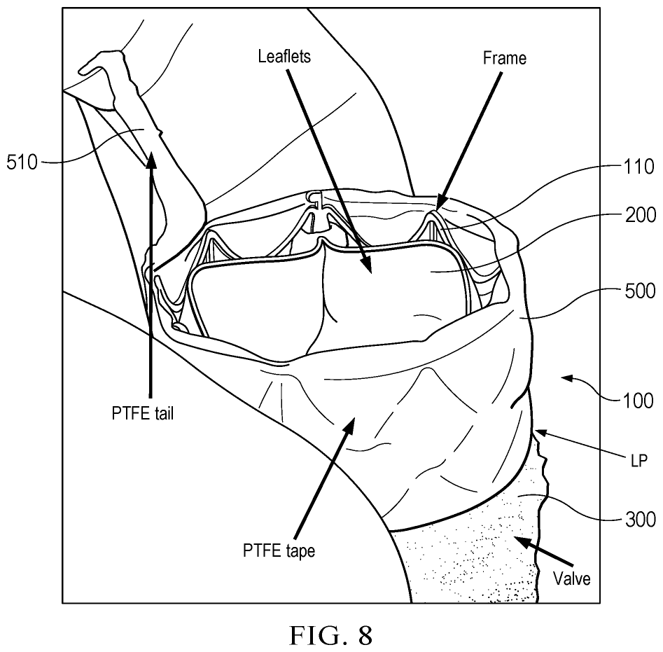

After the final crimping of the prosthetic valve 100 on the catheter C or inflatable balloon B, the temporary/removeable barrier material 500 is removed from the prosthetic valve 100. When the temporary/removeable barrier material 500 includes the optional tail 510, handle, flap, tab, or grasping region, such optional tail 510, handle, flap, tab, or grasping region can be used by a user to facilitate in the removal of the temporary/removeable barrier material 500 from the prosthetic valve 100. FIG. 12 illustrates the prosthetic valve 100 after being crimped on the inflatable balloon B and the temporary/removeable barrier material 500 being removed from the prosthetic valve 100.

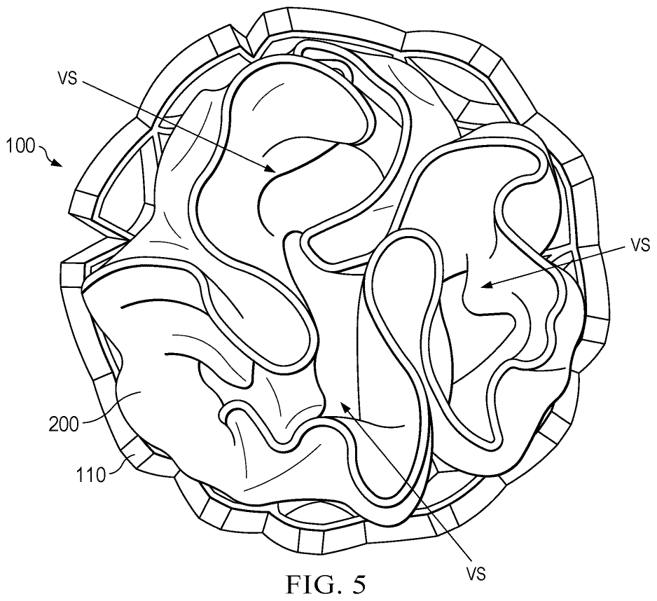

Referring now to FIG. 9, the leaflet protector LP is in the form of a temporary/removeable barrier material 500 and a foam sleeve 400 that are both positioned at least partially about the outer surface of the prosthetic valve 100. The temporary/removeable barrier material 500 is generally positioned about the prosthetic valve 100 is a similar manner as described above with regard to FIG. 8. The material and configuration of the temporary/removeable barrier material 500 is generally the as described above with regard to FIG. 8. The foam sleeve 400 is illustrated as being positioned about 50-100% (and all values and ranges therebetween) of the longitudinal length of the prosthetic valve 100. As illustrated in FIG. 9, the foam sleeve 400 has a longitudinal that is longer than the prosthetic valve 100; however, this is not required.

After the partial crimping of the prosthetic valve 100, the foam sleeve 400 is generally removed and the temporary/removeable barrier material 500 remains on at least a portion of the outer surface of the prosthetic valve 100 during the optional further crimping of the prosthetic valve 100. The foam sleeve 400 and the temporary/removeable barrier material 500 are used to inhibit or prevent one or more portions of one or more of the leaflets of leaflet structure 200 from moving into and be damaged in the smaller openings in the frame 110 during the crimping of the prosthetic valve 100.

When the temporary/removeable barrier material 500 is used in conjunction with the foam sleeve 400, the foam sleeve 400 is positioned about a portion or all of the prosthetic valve 100 after the temporary/removeable barrier material 500 has been wrapped about a portion or all of the prosthetic valve 100. Generally, the foam sleeve 400 is positioned about 70-100% (and all values and ranges therebetween) of the longitudinal length of the frame 110 that includes the temporary/removeable barrier material 500, and about 30-100% (and all values and ranges therebetween) of the longitudinal length of the prosthetic valve 100. As illustrated in FIG. 9, the foam sleeve 400 is positioned about 90-100% of the length of the prosthetic valve 100. As also illustrated in FIG. 9, the foam sleeve 400 can optionally extend beyond the proximal and/or distal ends of the prosthetic valve 100.

The foam sleeve 400 can have a generally cylindrical shape as illustrated in FIG. 9; however, it can be appreciated that the foam sleeve 400 can have other shapes. The foam sleeve 400 includes a cavity that extends along 70-100% (and all values and ranges therebetween) the longitudinal length of the foam sleeve 400. The diameter and/or cross-sectional area of the cavity is selected to be able to receive the prosthetic valve 100 without damaging the prosthetic valve 100. The thickness of the walls of the foam sleeve 400 is generally at least 2 mils (e.g., 2-40 mils and all values and ranges therebetween).

As illustrated in FIG. 9, the thickness of the wall of the foam sleeve 400 is greater than the thickness of the layer of temporary/removeable barrier material 500. The material used to form the foam sleeve 400 is generally a polymer material (e.g., polyethylene foam, polyurethane foam, neoprene foam, latex foam, memory foam, rebound foam, and conductive foam, etc.). Generally, the material used to form the foam sleeve 400 is different from the material used to form the temporary/removeable barrier material 500. Generally, the foam sleeve 400 is formed of a more compressible material than the material used to form the temporary/removeable barrier material 500. In one non-limiting embodiment, the material used to form the temporary/removeable barrier material 500 is configured to compress in thickness no more than 10% (e.g., 0-10% and all values and ranges therebetween) during the crimping of the prosthetic valve 100, and the material used to form the foam sleeve 400 is configured to compress in thickness at least 15% (e.g., 15-500% and all values and ranges therebetween) during the crimping of the prosthetic valve 100.

After the foam sleeve 400 is placed about the prosthetic valve 100 that includes the temporary/removeable barrier material 500, the prosthetic valve 100, the foam sleeve 400, and the temporary/removeable barrier material 500 are placed in the crimper 50 to crimp the prosthetic valve 100. The prosthetic valve 100 can optionally be positioned on the catheter C when the prosthetic valve is initially crimped. During the crimping of the prosthetic valve 100, the foam sleeve 400 is caused to be compressed as the crimper 50 applies an outer crimping force to the prosthetic valve 100. During the compression of the foam sleeve 400, portions of the foam sleeve 400 move toward, into and/or through one or more openings of the frame 110 as the frame 110 is being crimped. The movement of the portions of the foam sleeve 400 toward, into and/or through the openings of the frame 110 causes portions of the temporary/removeable barrier material 500 to also be pushed into and/or through one or more openings in the frame 110 as the frame 110 is being crimped.

After the crimping of the prosthetic valve 100 with the foam sleeve 400 is completed, the prosthetic valve 100 is removed from the crimper 50 and thereafter the foam sleeve 400 is removed from the prosthetic valve 100. After the foam sleeve 400 is removed from the prosthetic valve 100, the prosthetic valve 100 can optionally be again be placed into the crimper 50 while the temporary/removeable barrier material 500 remains on the prosthetic valve 100 for further crimping of the prosthetic valve 100. It can be appreciated that the prosthetic valve 100 can be further crimped in the crimper 50 while on the catheter C. The prosthetic valve 100 can be placed over the inflatable balloon B for final crimping of the prosthetic valve 100, or be positioned rearwardly of the inflatable balloon B for further crimping prior to be moved over the inflatable balloon B for final crimping of the prosthetic valve 100. During the further crimping of the prosthetic valve after the foam sleeve 400 is removed, the temporary/removeable barrier material 500 can remain on the prosthetic valve during further crimping of the prosthetic valve or be removed from the prosthetic valve prior to the final crimping of the prosthetic valve.

Referring now to FIG. 10, after the foam sleeve 400 is removed from the prosthetic valve 100, and after crimping of the prosthetic valve 100 with the temporary/removeable barrier material 500, the temporary/removeable barrier material 500 is illustrated as remaining about the frame 110 and is shown to be pushed from the outside of the frame 110 partially or fully through the openings in the frame 110. The movement of the temporary/removeable barrier material 500 partially into or fully through the openings in the frame 110 results in the one or more of the leaflets of leaflet structure 200 being pushed away from the openings in the frame 110 during the crimping of the prosthetic valve 100.

Referring again to FIG. 11, the prosthetic valve 100 is illustrated as being fully crimped and crimped about the inflatable balloon B, and little or none of the one or more of the leaflets of leaflet structure 200 are positioned in the openings in the frame 110 after the crimping of the prosthetic valve 100.

After the final crimping of the prosthetic valve 100 on the catheter C or inflatable balloon B, the temporary/removeable barrier material 500 is removed from the prosthetic valve 100. When the temporary/removeable barrier material 500 includes an optional tail 510, handle, flap, tab, or grasping region, such optional tail 510, handle, flap, tab, or grasping region can be used to facilitate in the removal of the temporary/removeable barrier material 500 from the prosthetic valve 100. FIG. 12 illustrates the prosthetic valve 100 after being crimped on the inflatable balloon B and the temporary/removeable barrier material 500 has been thereafter removed from the prosthetic valve 100.

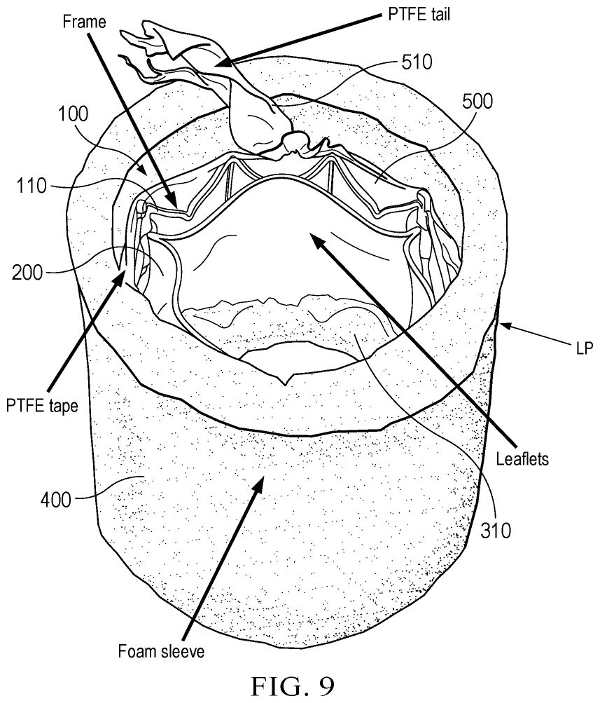

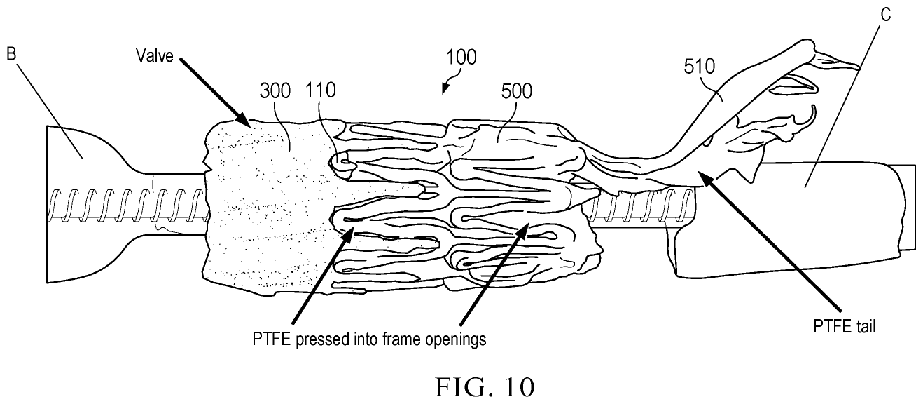

Referring now to FIGS. 13 and 14, an alternative embodiment of a leaflet protector LP is illustrated. The leaflet protector LP is in the form of a compression sleeve 400 that has a cavity 410 therethrough and one or more layers of a temporary/removeable barrier material 500 are positioned in the cavity 410 prior to the leaflet protector LP being positioned about the prosthetic valve 100. The temporary/removeable barrier material 500 optionally includes one or more optional tails 510, handles, flaps, tabs, or grasping regions extending out from the cavity 410 of the compression sleeve 400. The temporary/removeable barrier material 500 may or may not be connected to the compression sleeve 400. In one non-limiting configuration, the temporary/removeable barrier material 500 is not connected to the compression sleeve 400. In another non-limiting configuration, the temporary/removeable barrier material 500 is permanently or releasably connected to the compression sleeve 400 (e.g., adhesive connection, melted connection, stitched connection, staple connection, hook and loop fastener connection, mechanical connection, magnetic connection, etc.).

In one non-limiting configuration, the temporary/removeable barrier material 500 is removably positioned in cavity 410 of the compression sleeve 400. In such an arrangement, the one or more optional tails 510, handles, flaps, tabs, or grasping regions can optionally be used to facilitate in temporarily retaining or holding the temporary/removeable barrier material 500 in cavity 410. The temporary/removeable barrier material 500 can be positioned along 30-100% (and all values and ranges therebetween) the longitudinal length of cavity 410. When the temporary/removeable barrier material 500 is only positioned partially along the longitudinal length of cavity 410, the one or more optional tails 510, handles, flaps, tabs, or grasping regions, when used, may only extend out of one end of the compression sleeve 400; however, this is not required. When the temporary/removeable barrier material 500 is positioned along 90-100% of the longitudinal length of cavity 410, the one or more optional tails 510, handles, flaps, tabs, or grasping regions, when used, may extend out of one or both ends of the compression sleeve 400; however, this is not required.

In use, the leaflet protector LP is positioned about the prosthetic valve 100 prior to the crimping of the prosthetic valve 100. After the initial crimping of the prosthetic valve 100, the prosthetic valve 100 is removed from the crimper 50 and the compression sleeve 400 is removed from the prosthetic valve 100. If the temporary/removeable barrier material 500 is removably connected to the compression sleeve 400, the temporary/removeable barrier material 500 can optionally remain on a portion or all of the outer surface of the prosthetic valve 100 during further crimping of the prosthetic valve 100. However, if the temporary/removeable barrier material 500 is not removably connected to the compression sleeve 400, the removal of the compression sleeve 400 from the prosthetic valve 100 also result in the removal of the temporary/removeable barrier material 500 from the prosthetic valve 100 prior to any further crimping of the prosthetic valve 100.

As discussed above, during the crimping of the prosthetic valve 100, the compression of the compression sleeve 400 causes the temporary/removeable barrier material 500 to move into and/or through one or more of the openings in the frame 110. After the compression sleeve 400 or after both the compression sleeve 400 and the temporary/removeable barrier material 500 are removed from the prosthetic valve 100, the prosthetic valve 100 can optionally be subjected to further crimping as discussed above. After the prosthetic valve 100 has been subjected to further or final crimping, the temporary/removeable barrier material 500, if still on the prosthetic valve, is removed from the prosthetic valve 100. When the temporary/removeable barrier material 500 includes one or more optional tails 510, handles, flaps, tabs, or grasping regions, such one or more optional tails 510, handles, flaps, tabs, or grasping regions can be used to facilitate in the removal of the temporary/removeable barrier material 500 from the prosthetic valve 100.

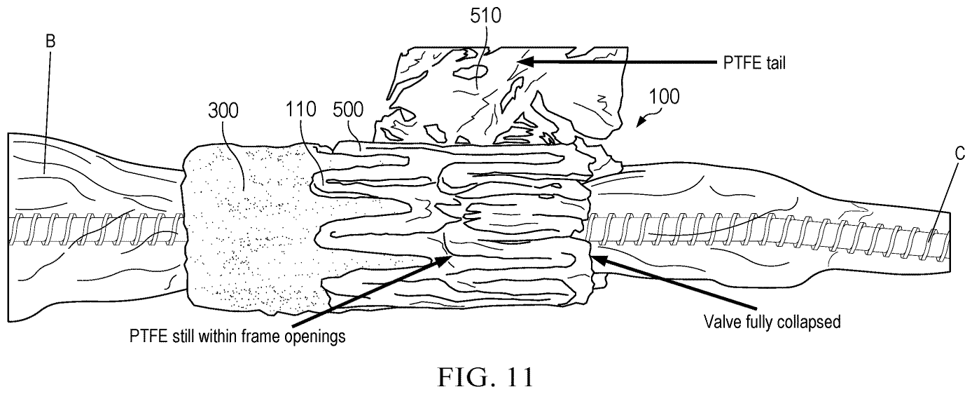

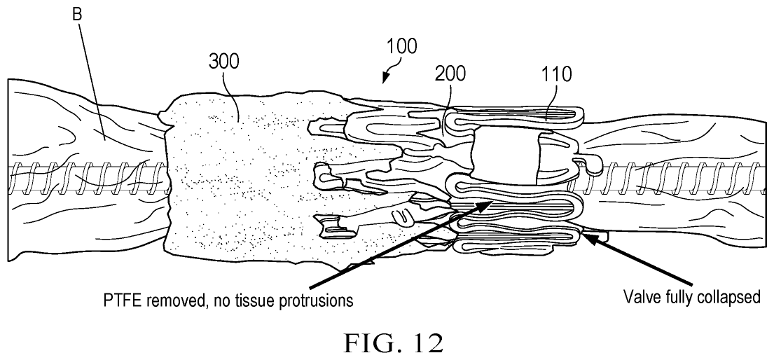

Referring now FIGS. 15 and 16, another non-limiting arrangement of a leaflet protector LP is illustrated wherein the leaflet protector LP includes one or more layers of flexible material 700 and a layer of compression material 600 that is permanently connected (e.g., adhesive connection, melted connection, heat compression connection, etc.) to a flexible material 700. The flexible material 700 can be positioned on one or both sides of the layer of compression material 600. One or more layers of flexible material 700 can be positioned on each side of the layer of compression material 600.

FIG. 15 illustrates the leaflet protector LP formed of a layer of compression material 600 and on one side of the compression material 600 there is connected a layer of flexible material 700. The type of connection between the compression material 600 and the layer of flexible material 700 is non-limiting (e.g., adhesive, heat/compression connection process, etc.). Although a single layer of compression material 600 and a single layer of flexible material 700 are illustrated, it will be appreciated that more than one layer can be used for either or both of the compression material 600 and flexible material 700 to form the leaflet protector LP.

Referring now to FIG. 16, the leaflet protector LP is formed of a layer of compression material 600 and on each side of the compression material 600 is connected a layer of flexible material 700. The type of connection between the compression material 600 and the layer of flexible material 700 is non-limiting (e.g., adhesive, heat/compression connection process, etc.). As can be appreciated, each side of the compression material 600 can included one or more layers of flexible material 700, and one or more layers of compression material 600 can be used to form the leaflet protector LP.

In one non-limiting embodiment, the flexible material 700 is a flexible polymer tape. In one non-limiting configuration, the flexible material 700 is a non-sintered PTFE tape. The width of the layer of flexible material 700 is generally at least 0.1 inches (e.g., 0.1-2 inches and all values and ranges therebetween). The thickness of each layer of flexible material 700 is generally at least 1 mil inches (e.g., 1-20 mils and all values and ranges therebetween). In another non-limiting configuration, the layer of compression material 600 is generally a polymer material (e.g., polyethylene foam, polyurethane foam, neoprene foam, latex foam, memory foam, rebound foam, and conductive foam, etc.). Generally, the material used to form the layer of compression material 600 is different from the material used to form the flexible material 700. The width of each layer of compression material 600 is generally the same or ±10% (and all values and ranges therebetween) the width of the layer of flexible material 700. The thickness of each the layer of compression material 600 is generally greater than the thickness of each layer of flexible material 700. In one non-limiting configuration, the thickness of the layer of compression material 600 is generally at least 2 mils (e.g., 2-40 mils and all values and ranges therebetween), and the thickness of the flexible material is at least 0.1 mils. In one non-limiting configuration, the width of the leaflet protector LP, the width of the layer of compression material 600, and/or the width of the layer of flexible material 700 is constant along 50-100% of the longitudinal length of the leaflet protector LP. In another non-limiting configuration, the thickness of the leaflet protector LP, the width of the layer of compression material 600, and/or the width of the layer of flexible material 700 is constant along 50-100% of the longitudinal length of the leaflet protector LP. In one non-limiting embodiment, the material used to form the flexible material 700 is configured to compress in thickness no more than 10% (e.g., 0-10% and all values and ranges therebetween) during the crimping of the prosthetic valve 100, and the material used to form the compression material 600 is configured to compress in thickness at least 15% (e.g., 15-500% and all values and ranges therebetween) during the crimping of the prosthetic valve 100.

The leaflet protector LP illustrated in FIGS. 15 and 16 is configured to be wrapped about a portion or all of the outer surface of the prosthetic valve 100 prior to the partial or full crimping of the prosthetic valve 100. In one non-limiting configuration, the leaflet protector LP is wrapped about 50-100% (and all values and ranges therebetween) of the longitudinal length of the prosthetic valve 100. During the wrapping of the leaflet protector LP about a portion or all of the outer surface of the prosthetic valve 100, the side edges of the leaflet protector LP can abut against one another, slightly overlap one another, and/or be slightly spaced from one another (e.g., 0.001-5 mils of spacing and all values and ranges therebetween). The leaflet protector LP can be used by itself or in combination the temporary/removeable barrier material 500 and/or the foam sleeve 400 as described above during the crimping of the prosthetic valve 100.

In one non-limiting operation of use, the leaflet protector LP is used without the use of a separate temporary/removeable barrier material 500 during the crimping of the prosthetic valve 100. In such operation, the leaflet protector LP is wrapped about a portion or all of the outer surface of the prosthetic valve 100, and the side edges of the leaflet protector LP generally abut against one another or slightly overlap one another. A releasable adhesive, a sleeve, adhesive tape, etc. may optionally be used to maintain the leaflet protector LP in a wrapped state on the prosthetic valve 100 after being inserted on the prosthetic valve 100.

When the leaflet protector LP has a configuration as illustrated in FIG. 15, the side of the leaflet protector LP that includes the flexible material 700 may face toward or face away from the outer surface of the prosthetic valve 100 during the wrapping of the leaflet protector LP about a portion or all of the prosthetic valve 100. In one non-limiting configuration, the flexible material 700 faces toward the outer surface or frame of the prosthetic valve 100 during the wrapping of the leaflet protector LP about a portion or all of the prosthetic valve 100.

After the leaflet protector LP is wrapped about a portion or all of the outer surface of the prosthetic valve 100, the prosthetic valve 100 is inserted into a crimper 50 to be crimped. During the crimping of the prosthetic valve 100, the compression material 600 is compressed and a portion of the compression material 600 moves toward, enters into and/or passes through one or more openings in the frame 110. Depending on the configuration of the leaflet protector LP and the wrapping orientation of the leaflet protector LP on the prosthetic valve 100, a portion of the flexible material 700 may or may not enter into and/or passes through one or more openings in the frame 110 during the crimping of the prosthetic valve 100. For example, when the leaflet protector LP has a configuration as illustrated in FIG. 15, and the top surface of the flexible material 700 faces away from the prosthetic valve 100, little or none of the flexible material 700 will enter into and/or passes through one or more openings in the frame 110 during the crimping of the prosthetic valve 100. However, when the leaflet protector LP has a configuration as illustrated in FIG. 15, and the top surface of the flexible material 700 faces toward the prosthetic valve 100, at least a portion of the flexible material 700 will enter into and/or passes through one or more openings in the frame 110 during the crimping of the prosthetic valve 100.

When the leaflet protector LP has a configuration as illustrated in FIG. 16, at least a portion of the top surface of the flexible material 700 on one side of the leaflet protector LP that faces toward the prosthetic valve 100, may enter into and/or passes through one or more openings in the frame 110 during the crimping of the prosthetic valve 100. After the crimping of the prosthetic valve 100 is partially or fully completed, the leaflet protector LP is removed from the prosthetic valve 100. The prosthetic valve 100 can optionally be further crimped after removal of the leaflet protector LP.

In one non-limiting operation of use, the leaflet protector LP illustrated in FIGS. 15 and 16 is used with the temporary/removeable barrier material 500 during the crimping of the prosthetic valve 100. In such operation, one or more layers of temporary/removeable barrier material 500 are wrapped about a portion or all of the outer surface of the prosthetic valve 100. Thereafter, the leaflet protector LP illustrated in FIGS. 15 and 16 is wrapped about a portion or all of the outer surface of the prosthetic valve 100 and a portion of all of the outer surface of the temporary/removeable barrier material 500. During the wrapping of the LP, and the side edges of the leaflet protector LP generally abut against one another or slightly overlap one another. When the leaflet protector LP has a configuration as illustrated in FIG. 15, the side of the leaflet protector LP that includes the flexible material 700 may face toward or face away from the outer surface of the temporary/removeable barrier material 500 on the prosthetic valve 100 during the wrapping of the leaflet protector LP about a portion or all of the prosthetic valve 100. Generally, 60-100% (and all values and ranges therebetween) of the outer surface of the temporary/removeable barrier material 500 is covered by the leaflet protector LP after it has been wrapped about a portion or all of the outer surface of the prosthetic valve 100. A releasable adhesive, a sleeve, adhesive tape, etc. may optionally be used to maintain the leaflet protector LP in a wrapped state on the prosthetic valve 100 after being inserted on the prosthetic valve 100. After the leaflet protector LP is wrapped about a portion or all of the outer surface of the prosthetic valve 100, the prosthetic valve 100 is inserted into a crimper 50 to be crimped. During the crimping of the prosthetic valve 100, the compression material 600 is compressed and a portion of the compression material 600 moves toward, enters into and/or passes through one or more openings in the frame 110. Depending on the configuration of the leaflet protector LP, a portion of the flexible material 700 may or may not enter into and/or passes through one or more openings in the frame 110 during the crimping of the prosthetic valve 100. Also, during the crimping of the prosthetic valve 100, a portion of the temporary/removeable barrier material 500 is caused to enter into and/or passes through one or more openings in the frame 110 during the crimping of the prosthetic valve 100. After the crimping of the prosthetic valve 100 is partially or fully completed, the leaflet protector LP is removed from the prosthetic valve 100, and the temporary/removeable barrier material 500 may or may not remain on the prosthetic valve 100 for further crimping of the prosthetic valve 100. The prosthetic valve 100 can optionally be further crimped after removal of the leaflet protector LP. If crimping of the prosthetic valve 100 is completed after removal of the leaflet protector LP, then the temporary/removeable barrier material 500 is also removed from the prosthetic valve 100. If the prosthetic valve 100 is to be further crimped after removal of the leaflet protector LP, the temporary/removeable barrier material 500 can optionally remain on the prosthetic valve 100 during the further crimping of the prosthetic valve 100. Once final crimping of the prosthetic valve 100 is completed, and if the temporary/removeable barrier material 500 still remains on the prosthetic valve 100, the temporary/removeable barrier material 500 is then removed from the prosthetic valve 100.

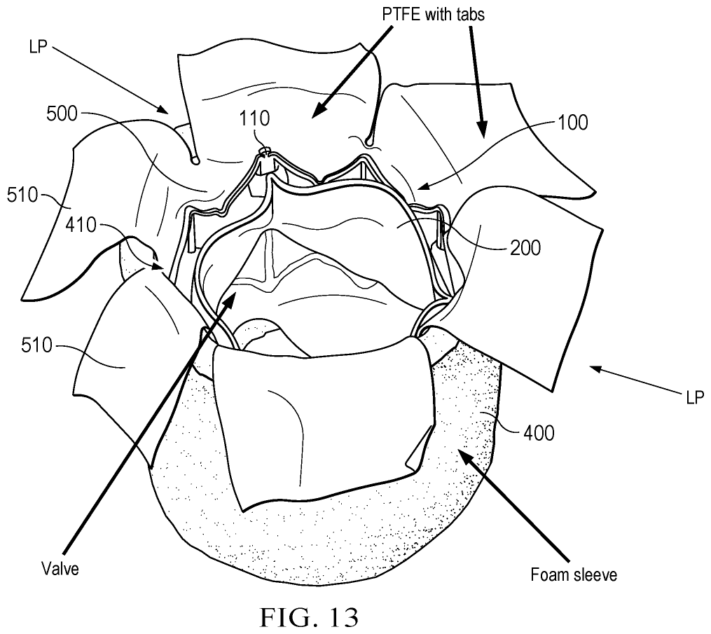

Referring now to FIGS. 17-22, another non-limiting embodiment of the leaflet protector LP is in the form of a temporary/removeable barrier material 500 that has been formed into the shape of a sleeve prior to be inserted about a portion or all of the outer surface of the prosthetic valve 100. The sleeve can be formed by extrusion or be formed from a strip temporary/removeable barrier material 500 that has been rolled or otherwise formed into the sleeve. Once the temporary/removeable barrier material 500 is formed in the shape of a sleeve and placed on a portion or all of the outer surface of the prosthetic valve 100, the prosthetic valve is then crimped as previously described above.

Referring again to FIG. 17, the temporary/removeable barrier material 500 is in the form of a strip 520 polymer material (e.g., non-sintered PTFE tape, etc.) having a length. Generally, the strip 520 of the polymer material is cut to a certain length of at least 0.25 inches (e.g., 0.25-5 inches and all values and ranges therebetween). A portion of the strip of polymer material can be optionally cut to form a smaller width portion 530 and a wider width portion 540. The wider width portion can be used to form the flap or tail 510 of the temporary/removeable barrier material 500. The width of the smaller width portion 530 is generally 5-70% smaller (and all values and ranges therebetween) than the wider width portion.

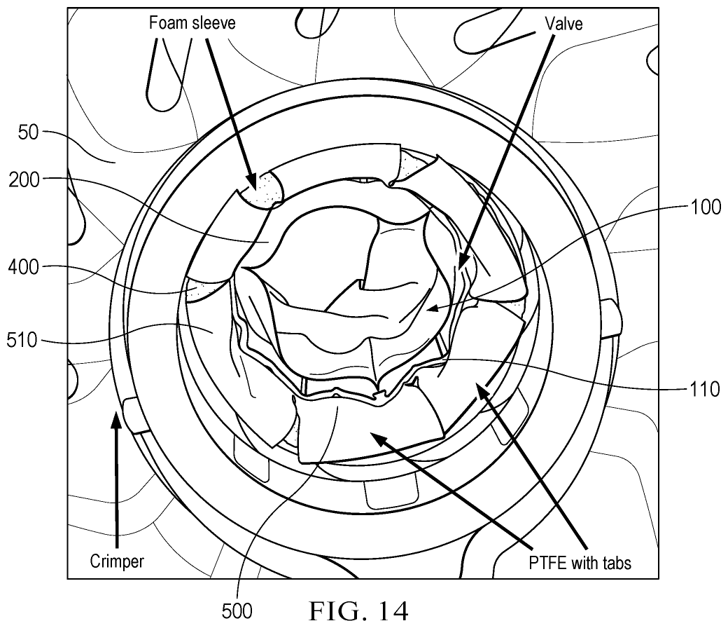

Referring now to FIG. 18, the strip 520 of polymer material is wound or wrapped one or more times about a mandrel M. A front portion 540 of strip 520 can optionally be located forwardly of the front end of mandrel M. Generally, the mandrel M has a cross-sectional area or diameter that is equal to or greater than the diameter of the uncrimped prosthetic valve (e.g., 1-1.5 times the cross-sectional area or diameter of the uncrimped prosthetic valve and all values and ranges therebetween).

In one non-limiting configuration, the front portion 540 of the wrapped strip 520 optionally extends off the front end of the mandrel M (e.g., 1-20% of a longitudinal length of the wrapped PTFE extends forwardly of the front end of the mandrel and all values and ranges therebetween). The longitudinal length of the strip 520 that is located rearwardly of the front end of the mandrel M (i.e., portion of the strip that is wound about the mandrel) can optionally be the desired longitudinal length of the wrapped strip that is to be positioned about the prosthetic valve 100 prior to the crimping of the prosthetic valve 100.

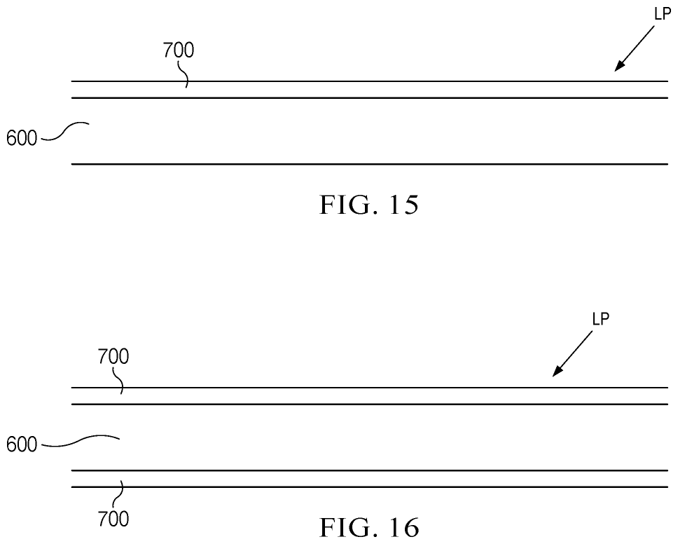

Referring now to FIG. 19, the portion of the strip 520 that is wrapped about the mandrel M can optionally be subjected to rolling by or on a textured surface to cause the outer surface of the strip 520 to create areas of increase the mechanical adherence 550 with the overlapping portions of the strip 520 on the mandrel M so as to improve the robustness and/or bonding together of the wrapped portions of the strip 520 that is located on the mandrel M.

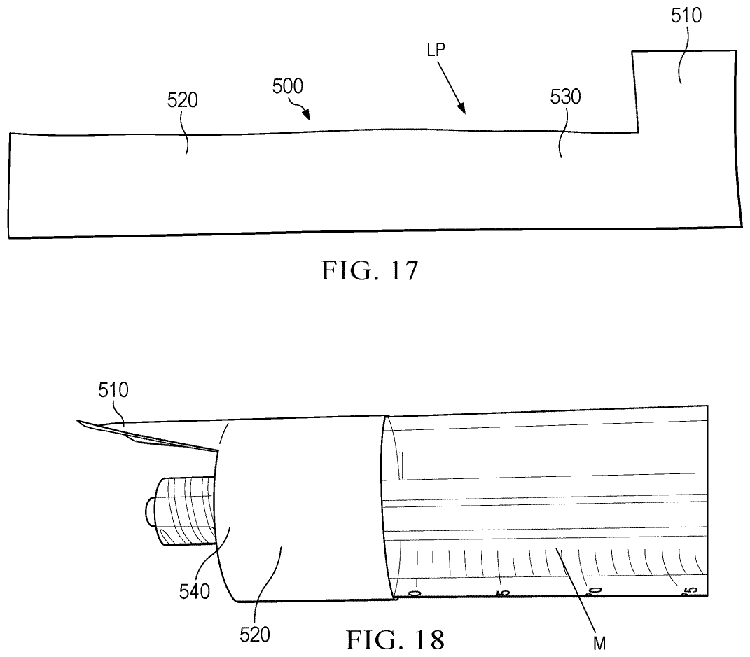

Referring now to FIG. 20, the front portion 540 of the strip 520 that extends forwardly of the front end of the mandrel M can optionally be pulled away from the front end of the mandrel M in a direction that is parallel or close to parallel to a longitudinal axis of the mandrel M so as to stretch the strip 520 that is located forwardly of the mandrel M in a direction that perpendicular or near perpendicular (0-20° to perpendicular and all values and ranges therebetween) to the direction of extrusion of the strip 520, thereby resulting in the strip 520 stretching in such direction. The front end of the mandrel M, during the stretching of the front portion of the strip 520, resists or prevents the stretching of the strip 520 that is positioned rearward of the front end of the mandrel M so as to create a wrapped strip that has a front portion that has a cavity having smaller diameter or cross-sectional area than the diameter or cross-sectional area of the cavity of the rear portion of the wrapped strip that formed by the outer surface of the mandrel M. When the strip 520 optionally includes a flap or tab 510, the flap or tab 510 is positioned at the front end portion of the strip 520. During the stretching of the strip 520, the stretching can optionally be in a heated environment (e.g., 90-200° F. and all values and ranges therebetween) to facilitate in the stretching of the strip 520. After the strip 520 is stretched, the strip 520 can optionally be slowly cooled (e.g., cooled at an average rate of 0.1-40° F./min. and all values and ranges therebetween) to ambient temperature (e.g., 60° F.-80° F.).

After the strip 520 has been stretched, the stretched strip is removed from the mandrel M. The removed stretched strip has a front portion 540 has a cavity having a smaller diameter or cross-sectional area than the cavity of the rear portion of the strip 520 that was positioned about the mandrel M so as to create a “hat shaped” strip assembly.

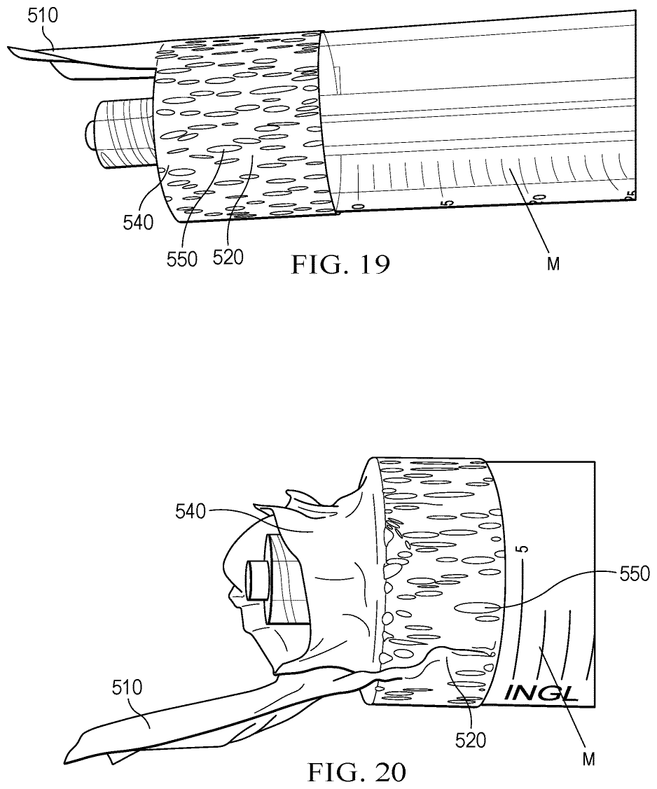

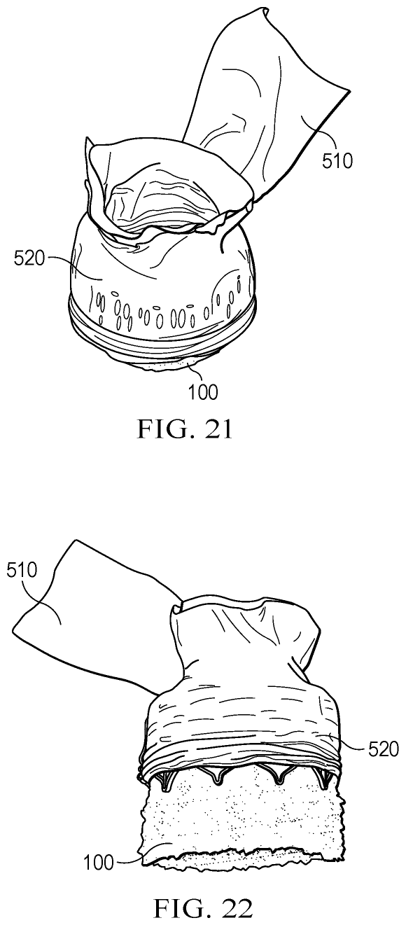

Referring now to FIGS. 21 and 22, the stretched strip assembly is positioned on the prosthetic valve 100 by inserting the rear portion of “hat” shaped strip assembly over a portion of the prosthetic valve 100. The smaller diameter or cross-sectional area front portion 540 of the strip assembly prevents the front portion 540 of the strip from being partially or fully positioned over or moved over the prosthetic valve 100, thereby ensuring that the designed length of the cavity on the rear portion of the strip assembly that has the larger cross-sectional area or diameter overlies the desired portion of the prosthetic valve 100.

After the prosthetic valve 100 is crimped, the strip assembly can be easily removed from the prosthetic valve by a user grasping the optional tap or flap 510 on the strip assembly and then pulling the strip assembly off of the crimped prosthetic valve. Even when the strip assembly has no tab or flap 510, the strip assembly can still be easily removed from the crimped prosthetic valve.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

What is claimed:

1. A method for reducing the outer diameter or cross-sectional area of a crimped prosthetic valve comprising:
providing a prosthetic valve; said prosthetic valve includes a frame, a leaflet structure supported by the frame; said frame is in a non-fully crimped orientation; said frame includes a plurality of frame openings;
providing a diameter reducing device that is configured to reduce an outer diameter or cross-sectional area of said frame of said prosthetic valve;
providing a leaflet protector that is configured to inhibit or prevent damage to said leaflet structure during crimping said frame of said prosthetic valve; said leaflet protector includes a layer of a flexible first polymer tape;
wrapping said layer of said first polymer tape at least one time about an outer surface of at least a portion of said frame;
at least partially inserting said prosthetic valve in said diameter reducing device;
reducing an outer diameter or cross-sectional area of said frame of said prosthetic valve by use of said diameter reducing device; and
removing said leaflet protector from said frame after said step of reducing said outer diameter or cross-sectional area of said prosthetic valve; and
wherein said leaflet protector inhibits or prevents said leaflet structure from moving into one or more of said plurality of openings in said frame as said outer diameter or cross-sectional area of said frame of said prosthetic valve is reduced; and
wherein said step of reducing causes at least a portion of said leaflet protector to enter into and/or pass through one or more of said plurality of openings in said frame.

2. The method as defined in claim 1, wherein said first polymer tape is non-sintered PTFE tape.

3. The method as defined in claim 1, wherein said layer of said first polymer tape is wrapped at least 1.1 times about at least a portion of said frame.

4. The method as defined in claim 1, wherein at least a portion of said first polymer tape is not wrapped about any portion of said prosthetic valve so as to form a tail on first polymer tape; said tail is configured to facilitate in removal of said first polymer tape from said frame after said outer diameter or cross-sectional area of said frame of said prosthetic valve has been reduced; said tail constitutes at least 2% of a longitudinal length of said first polymer tape.

5. The method as defined in claim 1, wherein 30-100% of a longitudinal length of said frame includes said first polymer tape wrapped about said outer surface of said frame.

6. The method as defined in claim 1, wherein said prosthetic valve includes an outer skirt; 0-20% of a longitudinal length of said outer skirt as measured along a longitudinal axis of said frame includes said first polymer tape wrapped about said outer surface of said outer skirt.

7. The method as defined in claim 1, wherein said leaflet protector includes said layer of said first polymer tape and a layer of compressible material; said layer of first polymer tape is located on a first side of said compressible material when said leaflet protector is positioned about said frame; said compressible material is formed of a different material from said first polymer tape; a thickness of said compressible material is greater than a thickness of said layer of said first polymer tape; a compressibility of said compressible material is greater than a compressibility of said layer of said first polymer tape.

8. The method as defined in claim 7, wherein said first polymer tape is connected to said first side of said layer of compressible material.

9. The method as defined in claim 7, wherein said first polymer tape is releasably positioned on and/or releasably connected to said first side of said layer of compressible material.

10. The method as defined in claim 7, wherein leaflet protector further includes a layer of a second polymer tape; said layer of said second polymer tape is positioned on a second side of said compressible material; said compressible material is formed of a different material from said second polymer tape; a thickness of said compressible material is greater than a thickness of said layer of said second polymer tape; a compressibility of said compressible material is greater than a compressibility of said layer of said second polymer tape; said second polymer tape is a) a same material as said layer of said first polymer tape, and/or b) a same thickness as said layer of said first polymer tape.

11. The method as defined in claim 10, wherein said second polymer tape is connected to said second side of said layer of compressible material.

12. The method as defined in claim 10, wherein said second polymer tape is releasably positioned on and/or releasably connected to said second side of said layer of compressible material.

13. The method as defined in claim 7, wherein said compressible material is a compressible foam sleeve; said compressible foam sleeve includes an internal cavity that extends at least 50% of a longitudinal length of said compressible foam sleeve; and further including the step of inserting said compressible foam sleeve about at least a portion of an outer surface of said prosthetic valve; at least a portion of said first polymer tape is positioned in said internal cavity of said compressible foam sleeve when said compressible foam sleeve is inserted about at least a portion of an outer surface of said prosthetic valve.

14. The method as defined in claim 7, further including the step of positioning said compressible foam sleeve about at least a portion of said outer surface of said first polymer tape after said first polymer tape has been applied to at least a portion of said outer surface of said frame.

15. The method as defined in claim 7, further including the steps of i) removing said compressible foam sleeve after said outer diameter or cross-sectional area of said prosthetic valve has been reduce; ii) subjecting said prosthetic valve to further diameter reduction or cross-sectional area reduction while said first polymer tape remains on said frame and after said compressible foam sleeve has been removed from said prosthetic valve; and iii) removing said first polymer tape from said frame after a further diameter reduction or cross-sectional reduction of said frame of said prosthetic valve.

16. A prosthetic valve system configured to cause an outer diameter or cross-sectional area of a prosthetic valve to be reduced while limiting damage to one or more components of said prosthetic valve; said prosthetic valve system comprising:
said prosthetic valve; said prosthetic valve includes a frame, and a leaflet structure supported by the frame; and
a leaflet protector that is configured to inhibit or prevent damage to said leaflet structure during crimping said frame of said prosthetic valve; said leaflet protector includes a flexible layer of first polymer tape; said layer of said first polymer tape is wrapped at least once about an outer surface of at least a portion of said frame prior to fully reducing said outer diameter or cross-sectional area of said frame of said prosthetic valve.

17. The prosthetic valve system as defined in claim 16, wherein said first polymer tape is non-sintered PTFE tape.

18. The prosthetic valve system as defined in claim 16, wherein said layer of said first polymer tape is wrapped at least 1.1 times about at least a portion of said frame.

19. The prosthetic valve system as defined in claim 16, wherein at least a portion of said first polymer tape is not wrapped about any portion of said prosthetic valve so as to form a tail on first polymer tape; said tail is configured to facilitate in removal of said first polymer tape from said frame after said outer diameter or cross-sectional area of said frame of said prosthetic valve has been reduced; said tail constitutes at least 2% of a longitudinal length of said first polymer tape.

20. The prosthetic valve system as defined in claim 16, wherein 30-100% of a longitudinal length of said frame includes said first polymer tape wrapped about said outer surface of said frame.

21. The prosthetic valve system as defined in claim 16, wherein said prosthetic valve includes an outer skirt; 0-20% of a longitudinal length of said outer skirt as measured along a longitudinal axis of said frame includes said first polymer tape wrapped about said outer surface of said outer skirt.

22. The prosthetic valve system as defined in claim 16, wherein said leaflet protector includes said layer of said first polymer tape and a layer of compressible material; said layer of first polymer tape is located on a first side of said compressible material when said leaflet protector is positioned about said frame; said compressible material is formed of a different material from said first polymer tape; a thickness of said compressible material is greater than a thickness of said layer of said first polymer tape; a compressibility of said compressible material is greater than a compressibility of said layer of said first polymer tape.

23. The prosthetic valve system as defined in claim 22, wherein said first polymer tape is connected to said first side of said layer of compressible material.

24. The prosthetic valve system as defined in claim 22, wherein said first polymer tape is releasably positioned on and/or releasably connected to said first side of said layer of compressible material.

25. The prosthetic valve system as defined in claim 22, wherein leaflet protector further includes a layer of a second polymer tape; said layer of said second polymer tape is positioned on a second side of said compressible material; said compressible material is formed of a different material from said second polymer tape; a thickness of said compressible material is greater than a thickness of said layer of said second polymer tape; a compressibility of said compressible material is greater than a compressibility of said layer of said second polymer tape; said second polymer tape is a) a same material as said layer of said first polymer tape, and/or b) a same thickness as said layer of said first polymer tape.

26. The prosthetic valve system as defined in claim 25, wherein said second polymer tape is connected to said second side of said layer of compressible material.

27. The prosthetic valve system as defined in claim 25, wherein said second polymer tape is releasably positioned on and/or releasably connected to said second side of said layer of compressible material.

28. The prosthetic valve system as defined in claim 22, wherein said compressible material is a compressible foam sleeve; said compressible foam sleeve includes an internal cavity that extends at least 50% of a longitudinal length of said compressible foam sleeve; and further including the step of inserting said compressible foam sleeve about at least a portion of an outer surface of said prosthetic valve; at least a portion of said first polymer tape is positioned in said internal cavity of said compressible foam sleeve when said compressible foam sleeve is inserted about at least a portion of an outer surface of said prosthetic valve.

29. The prosthetic valve system as defined in claim 28, further including the step of positioning said compressible foam sleeve about at least a portion of said outer surface of said first polymer tape after said first polymer tape has been applied to at least a portion of said outer surface of said frame.

30. The prosthetic valve system as defined in claim 28, further including the steps of i) removing said compressible foam sleeve after said outer diameter or cross-sectional area of said prosthetic valve has been reduce; ii) subjecting said prosthetic valve to further diameter reduction or cross-sectional area reduction while said first polymer tape remains on said frame and after said compressible foam sleeve has been removed from said prosthetic valve; and iii) removing said first polymer tape from said frame after a further diameter reduction or cross-sectional reduction of said frame of said prosthetic valve.
